(19) United States
(12) Patent Application Publication
(10) Pub. No.: US 2018/0002688 A1
(43) Pub. Date: Jan. 4, 2018

(54) METHODS FOR TAGGING DNA-ENCODED LIBRARIES

(71) Applicant: X-Chem, Inc., Waltham, MA (US)

(72) Inventors: Anthony D. KEEFE, Cambridge, MA (US); Alexander LITOVCHICK, Sudbury, MA (US); Matthew CLARK, Lexington, MA (US); Richard W. WAGNER, Cambridge, MA (US)

(73) Assignee: X-Chem, Inc., Waltham, MA (US)

(21) Appl. No.: 15/540,284

(22) PCT Filed: Dec. 28, 2015
(86) PCT No.: PCT/US2015/067667

§ 371 Date: Jun. 28, 2017

Related U.S. Application Data
(60) Provisional application No. 62/098,037, filed on Dec. 30, 2014.

(51) Int. Cl. C12N 15/10

(52) U.S. Cl. CPC C12N 15/1065

(57) ABSTRACT

The present invention relates to methods for producing encoded chemical entities. In particular, the oligonucleotides and methods can include encoded chemical entities having wild-type linkages formed through chemical ligation techniques. One strategy that can be utilized that simultaneously takes advantage of chemical ligation as a means to encode chemical history, while also retaining the ability of polymerases to directly recover tag sequence and association information, is to perform chemical ligation in a manner that generates wildtype phosphodiester linkages. Such methods generally utilize condensing agents such as cyanogen bromide or similar along with 5′-phosphate and 3′-hydroxyl oligonucleotides in a double-stranded or templated context. Similarly cyanogen bromide has also been shown to chemically ligate pairs of substrate oligonucleotides that are 5′-hydroxyl and 3′-phosphate. However, these methods suffer from poor efficiency making them ill-suited for use in an iterative process such as tagging DNA-en-coded libraries.

### BACKGROUND OF THE INVENTION

DNA-Encoded chemical library members are chemical entities generated by combinatorial chemical synthesis processes that are associated with combinations of encoding oligonucleotide tags. The combinations of tags associated with individual library members may be determined and used to deduce the chemical synthesis history of the associated library member.

One method to generate such libraries is by the successive chemical ligation of oligonucleotide tags to a headpiece oligonucleotide, upon which a chemically-generated entity is displayed, by successive split-and-mix steps. During each split step a chemical synthesis step is performed along with an oligonucleotide ligation step.

Oligonucleotide ligation steps that are chemical, rather than enzyme-mediated, permit greater flexibility with regard to solution conditions and may reduce the buffer exchange steps necessary—potentially for thousands of low-volume individually segregated compartments.

Most oligonucleotide linkage structures resulting from chemical ligation reactions, however, result in linkages that cannot be translocated through by polymerases. This means that such linkages cannot be utilized directly in processes that use polymerases to decode individual library members such as sequencing.

This invention relates to methods for tagging DNA-encoded chemical entities with wild-type linkages utilizing chemical ligation techniques. This allows for the advantages of chemical ligation to be realized while maintaining the convenience of linkages readable by polymerases.

### SUMMARY OF THE INVENTION

One strategy that can be utilized that simultaneously takes advantage of chemical ligation as a means to encode chemical history, while also retaining the ability of polymerases to directly recover tag sequence and association information, is to perform chemical ligation in a manner that generates wild-type phosphodiester linkages. Such methods generally utilize condensing agents such as cyanogen bromide or similar along with 5′-phosphate and 3′-hydroxyl oligonucleotides in a double-stranded or templated context. Similarly cyanogen bromide has also been shown to chemically ligate pairs of substrate oligonucleotides that are 5′-hydroxyl and 3′-phosphate. However, these methods suffer from poor efficiency making them ill-suited for use in an iterative process such as tagging DNA-encoded libraries.

The present inventors have developed an oligonucleotide tagging strategy that utilizes wild-type linkages (e.g., phosphodiester linkages) derived from oligonucleotide pairs bearing 5′-monophospho and 3′-hydroxy termini and also from 5′-hydroxy and 3′-monophospho termini using cyanoimidazole and Zn²⁺ in relatively high yields. Because this chemical ligation method is template-dependent and permits the use of orthogonal 3′- and 5′-phosphates a high degree of control over the sequential ligation of encoding oligonucleotides with a low rate of misincorporation or miscoding may be exerted, including in a double-stranded context.

Accordingly, in a first aspect, the invention features a method of producing an encoded chemical entity. This method includes: (a) providing a headpiece comprising a first functional group and a second functional group; (b) binding the first functional group of the headpiece to a component of a chemical entity, wherein the headpiece is directly connected to the component or the headpiece is indirectly connected to the component by a bifunctional spacer; and (c) ligating the second functional group of the headpiece to a first oligonucleotide tag via chemical ligation to form an encoded chemical entity, wherein the chemical ligation generates a phosphodiester, phosphonate, or phosphorothioate linkage; wherein steps (b) and (c) can be performed in any order and wherein the first oligonucleotide tag encodes for the binding reaction of step (b), thereby producing an encoded chemical entity.

In another aspect, the invention features a further method of producing an encoded chemical entity. This method includes: (a) providing a headpiece comprising a first functional group and a second functional group; (b) binding the first functional group of the headpiece to a component of the chemical entity, wherein the headpiece is directly connected to the component or the headpiece is indirectly connected to the component by a bifunctional spacer; (c) ligating the second functional group of the headpiece to a first oligonucleotide tag via chemical ligation to form a complex, wherein the chemical ligation generates a phosphodiester, phosphonate, or phosphorothioate linkage; (d) binding n_{c} number of additional components of the encoded chemical entity, wherein n_{c} is an integer between 1 and 10; and (e) ligating nₜ number of additional oligonucleotide tags having nₜ linkages to form an encoded chemical entity wherein nₜ is an integer between 1 and 10 and wherein each of the linkages is between two adjacent tags and each tag encodes the identity of at least one of the components; wherein steps (b) and (c) can be performed in any order and wherein the first oligonucleotide tag encodes for the binding reaction of step (b); and wherein the steps (d) and (e) can be performed in any order and wherein each additional tag encodes for the binding reaction of each additional component of the step (d), thereby producing an encoded chemical entity.

In some embodiments, ligation of at least one of nₜ linkages is not via chemical ligation that generates a phosphodiester, phosphonate, or phosphorothioate linkage (e.g., ligation of at least one of nₜ linkages is via enzymatic ligation, or chemical ligation that generates a readable or unreadable linkage).

In some embodiments, n_{c} and nₜ are each independently an integer between 1 and 2, 1 and 3, 1 and 4, 1 and 5, 1 and 6, 1 and 7, 1 and 8, 1 and 9, 1 and 10, 2 and 3, 2 and 4, 2 and 5, 2 and 6, 2 and 7, 2 and 8, 2 and 9, 2 and 10, 3 and 4, 3 and 5, 3 and 6, 3 and 7, 3 and 8, 3 and 9, 3 and 10, 4 and 5, 4 and 6, 4 and 7, 4 and 8, 4 and 9, 4 and 10. In certain embodiments, n_{c} is 1, 2, 3, 4, 5, 6, 7, 8, 9, or 10. In some embodiments, nₜ is 1, 2, 3, 4, 5, 6, 7, 8, 9, or 10.

In some embodiments, the chemical ligation generates a phosphodiester linkage. In certain embodiments, the chemical ligation generates a phosphonate linkage. In some embodiments, the chemical ligation generates a phosphorothioate linkage.

In some embodiments, the headpiece comprises a double-stranded oligonucleotide, a single-stranded oligonucleotide, or a hairpin oligonucleotide. In certain embodiments, the headpiece comprises a double stranded oligonucleotide or a hairpin oligonucleotide.

In some embodiments, the headpiece comprises a third functional group. In certain embodiments, the method further includes (d) ligating the third functional group of the headpiece to a second oligonucleotide tag via chemical ligation, wherein the chemical ligation generates a phosphodiester, phosphonate, or phosphorothioate linkage.

In some embodiments, the method further comprises (d) ligating said third functional group of said headpiece to a second oligonucleotide tag, wherein said ligation is not via chemical ligation that generates a phosphodiester, phosphonate, or phosphorothioate linkage (e.g., the ligation is enzymatic ligation or chemical ligation that results in a readable or unreadable linkage).

In certain embodiments, the headpiece includes a phosphate at the 5′-terminus and/or the 3′-terminus (e.g., the headpiece includes a phosphate at the 5′-terminus, the 3′-terminus, or when the headpiece is double stranded or a hairpin oligonucleotide, the headpiece optionally includes a phosphate on both the 5′-terminus and the 3′-terminus).

In some embodiments, the chemical ligation includes the ligation of a 5′- or 3′-phosphate on said headpiece to a 5′- or 3′-hydroxyl oligonucleotide. In some embodiments, the chemical ligation includes ligation of a phosphate at the 5′-terminus of the headpiece to a 5′-hydroxyl oligonucleotide or a 3′-hydroxyl oligonucleotide. In certain embodiments, the chemical ligation includes ligation of a phosphate at the 3-terminus of the headpiece to a 5′-hydroxyl oligonucleotide or a 3′-hydroxyl oligonucleotide.

In some embodiments, the chemical ligation includes the ligation of a 5′-phosphate on said headpiece to a 3′-hydroxyl oligonucleotide and/or a 3′-phosphate on said headpiece to a 5′-hydroxyl oligonucleotide. In some embodiments, the chemical ligation includes ligation of a phosphate at the 5′-terminus of the headpiece to a 3′-hydroxyl oligonucleotide and ligation of a phosphate at the 3′-terminus of the headpiece to a 5′-hydroxyl oligonucleotide.

In certain embodiments, the chemical ligation includes the simultaneous ligation of a 5′-phosphate on said headpiece to a 3′-hydroxyl oligonucleotide and a 3′-phosphate on said headpiece to a 5′-hydroxyl oligonucleotide.

In some embodiments, the chemical ligation includes the use of cyanoimidazole. In certain embodiments, the chemical ligation further includes the use of a divalent metal source (e.g., a soluble divalent metal source) such as a Zn²⁺ source (e.g., any soluble Zn²⁺ source such as ZnF₂, ZnCl₂, ZnBr₂, ZnI₂, Zn(NO₃)₂, Zn(ClO₃)₂, ZnSO₄, or Zn(O₂CCH₃)₂ or elemental zince oxidized in situ), a Mn²⁺ source (e.g., any soluble Mn²⁺ source such as MnSO₄, or MnCl₂), or a Co²⁺ source (e.g., any soluble Co²⁺ source such as CoF₂, CoCl₂, CoBr₂, or CoI₂).

In some embodiments, the headpiece is indirectly connected to the component of the chemical entity by a bifunctional spacer (e.g., linear or branched chains including a C₁₋₁₀ alkyl, a heteroalkyl of 1 to 10 atoms, a C₂₋₁₀ alkenyl, a C₂₋₁₀ alkynyl, C₅₋₁₀ aryl, a cyclic or a polycyclic system of 3 to 20 atoms, a phosphodiester, a peptide, an oligosaccharide, an oligonucleotide, an oligomer, a polymer, or a polyalkyl glycol such as a polyethylene glycol, such as —(CH₂CH₂O)ₙCH₂CH₂—, where n is an integer from 1 to 50).

In certain embodiments, the headpiece is directly connected to the component of an encoded chemical entity.

In certain embodiments, the chemical entity further includes one or more first library-identifying tag(s), use tag(s), and/or origin tag(s).

In some embodiments, the chemical entity includes between 2 to 20 tags (e.g., between 2 to 17 building block or scaffold tags, one first library-identifying tag, one optional use tag, and one origin tag). In some embodiments, each of the tags includes from 1 to 75 nucleotides (e.g., as described herein, such as about 6 to 12 nucleotides). In certain embodiments, each of the tags within an individual tag set includes about the same mass.

In some embodiments, the encoded chemical entity includes RNA, DNA, modified DNA, and/or modified RNA. In certain embodiments, the modified DNA or modified RNA is PNA, LNA, GNA, TNA, or a mixture thereof within the same oligonucleotide.

In certain embodiments, the encoded chemical entity includes a site for reversible immobilization. In some embodiments, the site for reversible immobilization is immobilized after at least one of the binding steps and released prior to a subsequent binding step. In some embodiments, the site for reversible immobilization is immobilized after a plurality of the binding steps and released prior to a subsequent binding step.

In some embodiments, the site for reversible immobilization includes one member of a binding pair, e.g., a nucleic acid, such as a hybridization-competent oligonucleotide (e.g., a hybridization-competent single-stranded oligonucleotide), a peptide, or a small molecule.

In another aspect, the invention features a library including one or more chemical entities produced by any of the foregoing methods.

In certain embodiments, the library includes a plurality of headpieces. In some embodiments, each headpiece of the plurality of headpieces includes an identical sequence region (e.g., a primer-binding region) and a different encoding region (e.g., a first tag that encodes for use of the library, origin of the library, identity of the library, history of the library, a linkage, a spacer, or addition of a first component; or an oligonucleotide sequence that facilitates hybridization, amplification, cloning, or sequencing technologies).

In certain embodiments, the library includes between about 10² to 10²⁰ chemical entities (e.g., about 10² to 10³, 10² to 10⁴, 10² to 10⁵, 10² to 10⁶, 10² to 10⁷, 10² to 10⁸, 10² to 10⁹, 10² to 10¹⁰, 10² to 10¹¹, 10² to 10¹², 10² to 10¹³, 10² to 10¹⁴, 10² to 10¹⁵, 10² to 10¹⁶, 10² to 10¹⁷, 10² to 10¹⁸, 10² to 10¹⁹, 10⁴ to 10⁵, 10⁴ to 10⁶, 10⁴ to 10⁷, 10⁴ to 10⁸, 10⁴ to 10⁹, 10⁴ to 10¹⁰, 10⁴ to 10¹¹, 10⁴ to 10¹², 10⁴ to 10¹³, 10⁴ to 10¹⁴, 10⁴ to 10¹⁵, 10⁴ to 10¹⁶, 10⁴ to 10¹⁷, 10⁴ to 10¹⁸, 10⁴ to 10¹⁹, 10⁴ to 10²⁰, 10⁵ to 10⁶, 10⁵ to 10⁷, 10⁵ to 10⁸, 10⁵ to 10⁹, 10⁵ to 10¹⁰, 10⁵ to 10¹¹, 10⁵ to 10¹², 10⁵ to 10¹³, 10⁵ to 10¹⁴, 10⁵ to 10¹⁵, 10⁵ to 10¹⁶, 10⁵ to 10¹⁷, 10⁵ to 10¹⁸, 10⁵ to 10¹⁹, or 10⁵ to 10²⁰ complexes). In certain embodiments of the library, each chemical entity is different.

In another aspect, the invention features a method of screening a plurality of encoded chemical entities. This method includes: (a) contacting a target with an encoded chemical entity prepared by any of the foregoing methods and/or any of the foregoing libraries; and (b) selecting one or more encoded chemical entities having a predetermined characteristic for said target, as compared to a control, thereby screening a plurality of encoded chemical entities.

In some embodiments, the predetermined characteristic includes increased binding for the target, as compared to a control. In certain embodiments, the predetermined characteristic includes increased inhibition of the target, as compared to a control. In some embodiments, the predetermined characteristic includes increased activity of the target, as compared to a control.

In any of the above embodiments, an oligonucleotide (e.g., the headpiece, the first tag, and/or one or more additional tags, if present) encodes for the identity of the library. In some embodiments, the oligonucleotide (e.g., the headpiece, the first tag, and/or one or more additional tags, if present) includes a first library-identifying sequence, where the sequence encodes for the identity of the first library. In particular embodiments, the oligonucleotide is a first library-identifying tag. In some embodiments, the method includes providing a first library-identifying tag, where the tag includes a sequence that encodes for a first library, and/or binding the first library-identifying tag to the complex. In some embodiments, the method includes providing a second library and combining the first library with a second library. In further embodiments, the method includes providing a second library-identifying tag, where the tag includes a sequence that encodes for a second library. In some embodiments, more than two libraries are combined (e.g., three, four, five, six, seven, eight, nine, ten, or more libraries).

In any of the above embodiments, the encoded information is provided in one or more tags or in a combination of more than one tag. In some embodiments, the encoded information is represented by more than one tag (e.g., two, three, four, five, six, seven, eight, nine, ten, or more tags). In some embodiments, the encoded information is represented by more than one tag, where all encoding tags are contained within the encoding sequence (e.g., by using of a specific tag combination to encode information). In some embodiments, the encoded information is represented by more than one tag, where less than all encoding tags are contained within the encoding sequence (e.g., by using one tag from a set of more than one individual tag to encode within an individual encoding sequence). In some embodiments, the encoded information is represented orthogonally, where encoded information is represented by a combination of more than one tag with less than all of the encoding information being contained within an individual library member, such that more than one corresponding library member needs to be sequenced in order to deconvolute the encoded information. In some embodiments, more than one chemical building block is represented by a single tag (e.g., for a racemic building block, such as two, three, four, five, six, seven, eight, nine, ten, or more building blocks represented by a single tag).

In any of the above embodiments, an oligonucleotide (e.g., a headpiece and/or one or more building blocks) encodes for the use of the member of the library (e.g., use in a selection step or a binding step, as described herein). In some embodiments, the oligonucleotide (e.g., the headpiece, the first tag, and/or one or more additional tags, if present) include a use sequence, where the sequence encodes for use of a subset of members in the library in one or more steps (e.g., a selection step and/or a binding step). In particular embodiments, the oligonucleotide is a use tag including a use sequence. In some embodiments, an oligonucleotide (e.g., a headpiece and/or one or more oligonucleotide tags) encodes for the origin of the member of the library (e.g., in a particular part of the library). In some embodiments, the oligonucleotide (e.g., the headpiece, the first tag, and/or one or more additional tags, if present) includes an origin sequence (e.g., a random or degenerate sequence having a length of about 10, 9, 8, 7, or 6 nucleotides), where the sequence enables the discrimination between amplification products derived from the same or different instances of otherwise identical library members. In particular embodiments, the oligonucleotide is an origin tag including an origin sequence. In some embodiments, the method further includes joining, binding, or operatively associating a use tag and/or an origin tag to the complex.

In any of the embodiments herein, the methods, compositions, and complexes optionally include a tailpiece, where the tailpiece includes one or more of a library-identifying sequence, a use sequence, or an origin sequence, as described herein. In particular embodiments, the methods further include joining, binding, or operatively associating the tailpiece (e.g., including one or more of a library-identifying sequence, a use sequence, or an origin sequence) to the complex.

In any of the above embodiments, the methods, compositions, and complexes, or portions thereof (e.g., the headpiece, the first tag, and/or the one or more additional tags, if present), may include a modification that support solubility in semi-, reduced-, or non-aqueous (e.g., organic) conditions. In some embodiments, the bifunctional spacer, headpiece, or one or more tags is modified to increase solubility of a member of said DNA-encoded chemical library in organic conditions In some embodiments, the modification is one or more of an alkyl chain, a polyethylene glycol unit, a branched species with positive charges, or a hydrophobic ring structure. In some embodiments, the modification includes one or more modified nucleotides having a hydrophobic moiety (e.g., modified at the C5 positions of T or C bases with aliphatic chains, such as in 5′-dimethoxytrityl-N4-diisobutylaminomethylidene-5-(1-propynyl)-2′-deoxycytidine,3′-[(2-cyanoethyl)-(N,N-diisopropyl)]-phosphoramidite; 5′-dimethoxytrityl-5-(1-propynyl)-2′-deoxyuridine,3′-[(2-cyanoethyl)-(N,N-diisopropyl)]-phosphoramidite; 5′-dimethoxytrityl-5-fluoro-2′-deoxyuridine,3′-[(2-cyanoethyl)-(N,N-diisopropyl)]-phosphoramidite; and 5′-dimethoxytrityl-5-(pyren-1-yl-ethynyl)-2′-deoxyuridine, or 3′-[(2-cyanoethyl)-(N,N-diisopropyl)]-phosphoramidite) or an insertion having a hydrophobic moiety (e.g., an azobenzene). In some embodiments, the member of the library has an octanol:water coefficient from about 1.0 to about 2.5 (e.g., about 1.0 to about 1.5, about 1.0 to about 2.0, about 1.3 to about 1.5, about 1.3 to about 2.0, about 1.3 to about 2.5, about 1.5 to about 2.0, about 1.5 to about 2.5, or about 2.0 to about 2.5).

In any of the above embodiments, a polymerase may have reduced ability to read or translocate through at least one of the linkages of an encoded chemical entity as described in International Application No. PCT/US13/50303, herein incorporated by reference. In some embodiments, the polymerase has reduced ability to read or translocate through at least about 10% (e.g., about 20%, 25%, 30%, 35%, 40%, 45%, 50%, 55%, 60%, 65%, 70%, 75%, 80%, 85%, 90%, 95%, 96%, 97%, 98%, 99%, or even 100%, as compared to control) of the linkages of an encoded chemical entity. In particular embodiments, the polymerase has reduced ability to read or translocate through about 10% to about 100% of the linkages of an encoded chemical entity (e.g., 20% to 100%, 25% to 100%, 50% to 100%, 75% to 100%, 90% to 100%, 95% to 100%, 10% to 95%, 20% to 95%, 25% to 95%, 50% to 95%, 75% to 95%, 90% to 95%, 10% to 90%, 20% to 90%, 25% to 90%, 50% to 90%, or 75% to 90%, as compared to control (e.g., as compared to a control oligonucleotide lacking the linkage)).

In some embodiments, less than about 10% (e.g., about 10%, 15%, 20%, 25%, 30%, 35%, 40%, 45%, 50%, 55%, 60%, 65%, 70%, 75%, 80%, 85%, 90%, or 95%) of the linkages of an encoded chemical entity include an enzymatic linkage. In some embodiments, the linkages of an encoded chemical entity include between 0% to 90% enzymatic linkages (e.g., about 0% to 40%, 0% to 45%, 0% to 50%, 0% to 55%, 0% to 60%, 0% to 65%, 0% to 70%, 0% to 75%, 0% to 80%, 0% to 85%, 0% to 90%, 0% to 95%, 0% to 96%, 0% to 97%, 0% to 98%, 0% to 99%, 5% to 40%, 5% to 45%, 5% to 50%, 5% to 55%, 5% to 60%, 5% to 65%, 5% to 70%, 5% to 75%, 5% to 80%, 5% to 85%, 5% to 90%, 5% to 95%, 5% to 96%, 5% to 97%, 5% to 98%, 5% to 99%, 10% to 40%, 10% to 45%, 10% to 50%, 10% to 55%, 10% to 60%, 10% to 65%, 10% to 70%, 10% to 75%, 10% to 80%, 10% to 85%, 10% to 90%, 10% to 95%, 10% to 96%, 10% to 97%, 10% to 98%, 10% to 99%, 15% to 40%, 15% to 45%, 15% to 50%, 15% to 55%, 15% to 60%, 15% to 65%, 15% to 70%, 15% to 75%, 15% to 80%, 15% to 85%, 15% to 90%, 15% to 95%, 15% to 96%, 15% to 97%, 15% to 98%, 15% to 99%, 20% to 40%, 20% to 45%, 20% to 50%, 20% to 55%, 20% to 60%, 20% to 65%, 20% to 70%, 20% to 75%, 20% to 80%, 20% to 85%, 20% to 90%, 20% to 95%, 20% to 96%, 20% to 97%, 20% to 98%, or 20% to 99%).

In some embodiments, at least one of the linkages of an encoded chemical entity includes a chemical linkage (e.g., a chemical-reactive group, a photo-reactive group, an intercalating moiety, or a cross-linking oligonucleotide). In particular embodiments, at least one (e.g., two, three, four, five, or more) chemical-reactive group, photo-reactive group, or intercalating moiety is present in a 5′-connector at or in proximity to the 5′-terminus of the tag and/or in a 3′-connector at or in proximity to the 3′-terminus of the tag. In other embodiments, the sequence of at least one of the 5′-connector is complementary to the sequence of the adjacent 3′-connector or identical or sufficiently similar to allow for hybridization to a complementary oligonucleotide. In some embodiments, at least 10% (e.g., about 20%, 25%, 30%, 35%, 40%, 45%, 50%, 55%, 60%, 65%, 70%, 75%, 80%, 85%, 90%, 95%, 96%, 97%, 98%, 99%, or even 100%) of the linkages of an encoded chemical entity are chemical linkages. In other embodiments, about 10% to about 100% of the linkages of an encoded chemical entity (e.g., 20% to 100%, 25% to 100%, 50% to 100%, 75% to 100%, 90% to 100%, 95% to 100%, 10% to 95%, 20% to 95%, 25% to 95%, 50% to 95%, 75% to 95%, 90% to 95%, 10% to 90%, 20% to 90%, 25% to 90%, 50% to 90%, or 75% to 90%) are chemical linkages.

In some embodiments, the chemical-reactive group is selected from a pair of an optionally substituted alkynyl group and an optionally substituted azido group; a pair of an optionally substituted diene having a 4 π-electron system and an optionally substituted dienophile or an optionally substituted heterodienophile having a 2 π-electron system; a pair of a nucleophile and a strained heterocyclyl electrophile; a pair of an optionally substituted amino group and an aldehyde or a ketone group; a pair of an optionally substituted amino group and a carboxylic acid group; a pair of an optionally substituted hydrazine and an aldehyde or a ketone group; a pair of an optionally substituted hydroxylamine and an aldehyde or a ketone group; a pair of a nucleophile and an optionally substituted alkyl halide; a platinum complex; an alkylating agent; or a furan-modified nucleotide.

In some embodiments, the photo-reactive group includes an intercalating moiety, a psoralen derivative, an optionally substituted cyanovinylcarbazole group (e.g., a 3-cyanovinylcarbazole group, such as 3-cyanovinylcarbazole-1′-β-deoxyriboside-5′-triphosphate), an optionally substituted vinylcarbazole group (e.g., a amidovinylcarbazole group, a carboxyvinylcarbazole group, or a C₂₋₇ alkoxycarbonylvinylcarbazole group, as described herein), an optionally substituted cyanovinyl group, an optionally substituted acrylamide group, an optionally substituted diazirine group, an optionally substituted benzophenone, or an optionally substituted azide group.

In some embodiments, the intercalating moiety is a psoralen derivative (e.g., psoralen, 8-methoxypsoralen, or 4-hydroxymethyl-4,5,8-trimethyl-psoralen (HMT-psoralen)), an alkaloid derivative (e.g., berberine, palmatine, coralyne, sanguinarine (e.g., iminium or alkanolamine forms thereof, or aristololactam-β-D-glucoside), an ethidium cation (e.g., ethidium bromide), an acridine derivative (e.g., proflavine, acriflavine, or amsacrine), an anthracycline derivative (e.g., doxorubicin, epirubicin, daunorubicin (daunomycin), idarubicin, and aclarubicin), or thalidomide.

In some embodiments, the chemical linkage includes the cross-linking oligonucleotide, where the sequence of at least five nucleotides at the 5′-terminus of the cross-linking oligonucleotide is complementary to the sequence of at least five nucleotides at the 3′-terminus of one or more tags or identical or sufficiently similar to allow for hybridization to a complementary oligonucleotide, and where the sequence of at least five nucleotides at the 3′-terminus of the cross-linking oligonucleotide is complementary to the sequence of at least five nucleotides at the 5′-terminus of one or more tags or identical or sufficiently similar to allow for hybridization to a complementary oligonucleotide. In particular embodiments, the 3′-terminus of one or more tags includes a 3′-connector. In particular embodiments, the 5′-terminus of one or more tags includes a 5′-connector.

In some embodiments, the 5′-terminus and/or 3′-terminus of the cross-linking oligonucleotide include a reversible co-reactive group (e.g., a cyanovinylcarbazole group, a cyanovinyl group, an acrylamide group, a thiol group, or a vinyl sulfone group, as described herein).

In some embodiments, the 3′-connector and/or 5′-connector includes a reversible co-reactive group (e.g., a cyanovinylcarbazole group, a cyanovinyl group, an acrylamide group, a thiol group, or a vinyl sulfone group, as described herein).

In any of the above embodiments, the headpiece, the tailpiece, the first tag, the one or more additional tags, the library-identifying tag, the use tag, and/or the origin tag, if present, may include from about 5 to about 75 nucleotides (e.g., from 5 to 7 nucleotides, from 5 to 8 nucleotides, from 5 to 9 nucleotides, from 5 to 10 nucleotides, from 5 to 11 nucleotides, from 5 to 12 nucleotides, from 5 to 13 nucleotides, from 5 to 14 nucleotides, from 5 to 15 nucleotides, from 5 to 16 nucleotides, from 5 to 17 nucleotides, from 5 to 18 nucleotides, from 5 to 19 nucleotides, from 5 to 20 nucleotides, from 5 to 30 nucleotides, from 5 to 40 nucleotides, from 5 to 50 nucleotides, from 5 to 60 nucleotides, from 5 to 70 nucleotides, from 6 to 7 nucleotides, from 6 to 8 nucleotides, from 6 to 9 nucleotides, from 6 to 10 nucleotides, from 6 to 11 nucleotides, from 6 to 12 nucleotides, from 6 to 13 nucleotides, from 6 to 14 nucleotides, from 6 to 15 nucleotides, from 6 to 16 nucleotides, from 6 to 17 nucleotides, from 6 to 18 nucleotides, from 6 to 19 nucleotides, from 6 to 20 nucleotides, from 7 to 8 nucleotides, from 7 to 9 nucleotides, from 7 to 10 nucleotides, from 7 to 11 nucleotides, from 7 to 12 nucleotides, from 7 to 13 nucleotides, from 7 to 14 nucleotides, from 7 to 15 nucleotides, from 7 to 16 nucleotides, from 7 to 17 nucleotides, from 7 to 18 nucleotides, from 7 to 19 nucleotides, from 7 to 20 nucleotides, from 8 to 9 nucleotides, from 8 to 10 nucleotides, from 8 to 11 nucleotides, from 8 to 12 nucleotides, from 8 to 13 nucleotides, from 8 to 14 nucleotides, from 8 to 15 nucleotides, from 8 to 16 nucleotides, from 8 to 17 nucleotides, from 8 to 18 nucleotides, from 8 to 19 nucleotides, from 8 to 20 nucleotides, from 9 to 10 nucleotides, from 9 to 11 nucleotides, from 9 to 12 nucleotides, from 9 to 13 nucleotides, from 9 to 14 nucleotides, from 9 to 15 nucleotides, from 9 to 16 nucleotides, from 9 to 17 nucleotides, from 9 to 18 nucleotides, from 9 to 19 nucleotides, from 9 to 20 nucleotides, from 10 to 11 nucleotides, from 10 to 12 nucleotides, from 10 to 13 nucleotides, from 10 to 14 nucleotides, from 10 to 15 nucleotides, from 10 to 16 nucleotides, from 10 to 17 nucleotides, from 10 to 18 nucleotides, from 10 to 19 nucleotides, from 10 to 20 nucleotides, from 10 to 30 nucleotides, from 10 to 40 nucleotides, from 10 to 50 nucleotides, from 10 to 60 nucleotides, from 10 to 70 nucleotides, from 10 to 75 nucleotides, from 11 to 12 nucleotides, from 11 to 13 nucleotides, from 11 to 14 nucleotides, from 11 to 15 nucleotides, from 11 to 16 nucleotides, from 11 to 17 nucleotides, from 11 to 18 nucleotides, from 11 to 19 nucleotides, from 11 to 20 nucleotides, from 12 to 13 nucleotides, from 12 to 14 nucleotides, from 12 to 15 nucleotides, from 12 to 16 nucleotides, from 12 to 17 nucleotides, from 12 to 18 nucleotides, from 12 to 19 nucleotides, from 12 to 20 nucleotides, from 13 to 14 nucleotides, from 13 to 15 nucleotides, from 13 to 16 nucleotides, from 13 to 17 nucleotides, from 13 to 18 nucleotides, from 13 to 19 nucleotides, from 13 to 20 nucleotides, from 14 to 15 nucleotides, from 14 to 16 nucleotides, from 14 to 17 nucleotides, from 14 to 18 nucleotides, from 14 to 19 nucleotides, from 14 to 20 nucleotides, from 15 to 16 nucleotides, from 15 to 17 nucleotides, from 15 to 18 nucleotides, from 15 to 19 nucleotides, from 15 to 20 nucleotides, from 16 to 17 nucleotides, from 16 to 18 nucleotides, from 16 to 19 nucleotides, from 16 to 20 nucleotides, from 17 to 18 nucleotides, from 17 to 19 nucleotides, from 17 to 20 nucleotides, from 18 to 19 nucleotides, from 18 to 20 nucleotides, from 19 to 20 nucleotides, from 20 to 30 nucleotides, from 20 to 40 nucleotides, from 20 to 50 nucleotides, from 20 to 60 nucleotides, from 20 to 70 nucleotides, from 20 to 75 nucleotides, from 30 to 40 nucleotides, from 30 to 50 nucleotides, from 30 to 60 nucleotides, from 30 to 70 nucleotides, from 30 to 75 nucleotides, from 40 to 50 nucleotides, from 40 to 60 nucleotides, from 40 to 70 nucleotides, from 40 to 75 nucleotides, from 50 to 60 nucleotides, from 50 to 70 nucleotides, from 50 to 75 nucleotides, from 60 to 70 nucleotides, from 60 to 75 nucleotides, and from 70 to 75 nucleotides). In particular embodiments, the headpiece, the first tag, the second tag, the one or more additional tags, the library-identifying tag, the use tag, and/or the origin tag, if present, have a length of less than 20 nucleotides (e.g., less than 19 nucleotides, less than 18 nucleotides, less than 17 nucleotides, less than 16 nucleotides, less than 15 nucleotides, less than 14 nucleotides, less than 13 nucleotides, less than 12 nucleotides, less than 11 nucleotides, less than 10 nucleotides, less than 9 nucleotides, less than 8 nucleotides, or less than 7 nucleotides).

In any of the above embodiments, the encoding sequence (e.g., the headpiece, the tailpiece, the first tag, the one or more additional tags, the library-identifying tag, the use tag, and/or the origin tag, if present) may include more than 20 nucleotides (e.g., more than 25, 30 35, 40, 45, 50, 55, 60, 65, 70, or 75 nucleotides).

### Definitions

By “about” is meant +/−10% of the recited value.

By “bifunctional” is meant having two reactive groups that allow for binding of two chemical moieties.

By “bifunctional spacer” is meant a spacing moiety having two reactive groups that allow for binding of a chemical entity and the encoding information of the complex. In one non-limiting example, the bifunctional spacer is provided between a chemical entity and a tag. In another non-limiting example, the bifunctional spacer is provided between a chemical entity and a headpiece. Exemplary bifunctional spacers are provided herein.

By “binding” is meant attaching by a covalent bond or a non-covalent bond. Non-covalent bonds include those formed by van der Waals forces, hydrogen bonds, ionic bonds, entrapment or physical encapsulation, absorption, adsorption, and/or other intermolecular forces. Binding can be effectuated by any useful means, such as by enzymatic binding (e.g., enzymatic ligation to provide an enzymatic linkage) or by chemical binding (e.g., chemical ligation to provide a chemical linkage). By “ligating” is meant attaching by a covalent bond.

By “building block” is meant a structural unit of a chemical entity, where the unit is directly linked to other chemical structural units or indirectly linked through the scaffold. When the chemical entity is polymeric or oligomeric, the building blocks are the monomeric units of the polymer or oligomer. Building blocks can have one or more diversity nodes that allow for the addition of one or more other building blocks or scaffolds. In most cases, each diversity node is a functional group capable of reacting with one or more building blocks or scaffolds to form a chemical entity. Generally, the building blocks have at least two diversity nodes (or reactive functional groups), but some building blocks may have one diversity node (or reactive functional group). Alternatively, the encoded chemical or binding steps may include several chemical components (e.g., multi-component condensation reactions or multi-step processes). Reactive groups on two different building blocks should be complementary, i.e., capable of reacting together to form a covalent or a non-covalent bond.

By “chemical entity” is meant a compound comprising one or more building blocks, one or more scaffolds, or a site for reversible immobilization. The chemical entity can be any small molecule, peptide, nucleic acid, peptide drug, or drug candidate designed or built to have one or more desired characteristics, e.g., capacity to bind a biological target, solubility, availability of hydrogen bond donors and acceptors, rotational degrees of freedom of the bonds, positive charge, negative charge, or site for reversible immobilization. In certain embodiments, the chemical entity can be reacted further as a bifunctional or trifunctional (or greater) entity.

By “chemical-reactive group” is meant a reactive group that participates in a modular reaction, thus producing a linkage. Exemplary reactions and reactive groups include those selected from a Huisgen 1,3-dipolar cycloaddition reaction with a pair of an optionally substituted alkynyl group and an optionally substituted azido group; a Diels-Alder reaction with a pair of an optionally substituted diene having a 4 π-electron system and an optionally substituted dienophile or an optionally substituted heterodienophile having a 2 π-electron system; a ring opening reaction with a nucleophile and a strained heterocyclyl electrophile; a splint ligation reaction with a phosphorothioate group and an iodo group; and a reductive amination reaction with an aldehyde group and an amino group, as described herein. By “complementary” is meant a sequence capable of hybridizing, as defined herein, to form secondary structure (a duplex or a double-stranded portion of a nucleic acid molecule). The complementarity need not be perfect but may include one or more mismatches at one, two, three, or more nucleotides. For example, complementary sequence may contain nucleobases that can form hydrogen bonds according to Watson-Crick base-pairing rules (e.g., G with C, A with T or A with U) or other hydrogen bonding motifs (e.g., diaminopurine with T, 5-methyl C with G, 2-thiothymidine with A, inosine with C, pseudoisocytosine with G). The sequence and its complementary sequence can be present in the same oligonucleotide or in different oligonucleotides.

By “complex” or “ligated complex” is meant a headpiece that is operatively associated with a chemical entity and/or one or more oligonucleotide tags by a covalent bond or a non-covalent bond. The complex can optionally include a bifunctional spacer between the chemical entity and the headpiece.

By “component” of a chemical entity is meant either a scaffold or a building block.

By “connector” of an oligonucleotide tag is meant a portion of the tag at or in proximity to the 5′- or 3′-terminus having a fixed sequence. A 5′-connector is located at or in proximity to the 5′-terminus of an oligonucleotide, and a 3′-connector is located at or in proximity to the 3′-terminus of an oligonucleotide. When present in a complex, each 5′-connector may be the same or different, and each 3′-connector may be the same or different. In an exemplary, non-limiting complex having more than one tags, each tag can include a 5′-connector and a 3′-connector, where each 5′-connector has the same sequence and each 3′-connector has the same sequence (e.g., where the sequence of the 5′-connector can be the same or different from the sequence of the 3′-connector). In another exemplary, non-limiting complex, the sequence of the 5′-connector is designed to be complementary, as defined herein, to the sequence of the 3′-connector (e.g., to allow for hybridization between 5′- and 3′-connectors). The connector can optionally include one or more groups allowing for a linkage (e.g., a linkage for which a polymerase has reduced ability to read or translocate through, such as a chemical linkage).

By “constant” or “fixed constant” sequence is meant a sequence of an oligonucleotide that does not encode information. Non-limiting, exemplary portions of a complex having a constant sequence include a primer-binding region, a 5′-connector, or a 3′-connector. The headpiece of the invention can encode information (thus, a tag) or alternatively not encode information (thus, a constant sequence). Similarly, the tailpiece of the invention can encode or not encode information.

By “cross-linking oligonucleotide” is meant an oligonucleotide that operatively associates, as defined herein, at a particular junction between two adjacent tags in a complex. In a non-limiting example, one terminus of the cross-linking oligonucleotide hybridizes to the 3′-connector of a first tag and the other terminus of the cross-linking oligonucleotide hybridizes to the 5′-connector of a second tag that is adjacent to the first tag. Exemplary, non-limiting embodiments of cross-linking oligonucleotides include those having one or more reactive groups (e.g., a chemical-reactive group, a photo-reactive group, an intercalating moiety, or a reversible co-reactive group, or any described herein) that operatively associates with adjacent tags or connectors of adjacent tags.

By “diversity node” is meant a functional group at a position in the scaffold or the building block that allows for adding another building block.

By “headpiece” is meant a chemical structure for library synthesis that is operatively linked to a component of a first chemical entity, to a tag, e.g., a starting oligonucleotide, and to a second chemical entity comprising a site for reversible immobilization. Optionally a headpiece may contain few or no nucleotides, but may provide a point at which they may be operatively associated. Optionally, a bifunctional spacer connects the headpiece to the component.

By “hybridize” is meant to pair to form a double-stranded molecule between complementary oligonucleotides, or portions thereof, under various conditions of stringency. (See, e.g., Wahl, G. M. and S. L. Berger (1987) Methods Enzymol. 152:399; Kimmel, A. R. (1987) Methods Enzymol. 152:507.) For example, high stringency hybridization can be obtained with a salt concentration ordinarily less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. High stringency hybridization temperature conditions will ordinarily include temperatures of at least about 30° C., 37° C., or 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In an alternative embodiment, hybridization will occur at 37° C. in 500 mM NaCl, 50 mM trisodium citrate, 1% SDS, 35% formamide, and 100 μg/ml denatured salmon sperm DNA (ssDNA). In a further alternative embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, high stringency salt concentrations for the wash steps may be, e.g., less than about 30 mM NaCl and 3 mM trisodium citrate, or less than about 15 mM NaCl and 1.5 mM trisodium citrate. High stringency temperature conditions for the wash steps will ordinarily include a temperature of, e.g., at least about 25° C., 42° C., or 68° C. In one embodiment, wash steps will occur at 25° C. in 30 mM NaCl, 3 mM trisodium citrate, and 0.1% SDS. In an alternative embodiment, wash steps will occur at 42° C. in 15 mM NaCl, 1.5 mM trisodium citrate, and 0.1% SDS. In a further alternative embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “intercalating moiety” is meant a reactive group that results in inclusion of a moiety between two or more nucleotides. In a non-limiting example, the intercalating moiety reacts with one or more nucleotides to form inter- or intra-strand cross-links between duplex or triplex oligonucleotides. Exemplary, non-limiting intercalating moieties are described herein.

By “junction” is meant a nick (lack of an internucleotide bond) or a gap (lack of one or more nucleotides) between two adjacent tags in a complex. The junction can also be between two adjacent connectors present in two adjacent tags (e.g., between the 3′-connector of a first tag and the 5′-connector of a second tag that is adjacent to the first tag).

By “library” is meant a collection of molecules or chemical entities. Optionally, the molecules or chemical entities are bound to one or more oligonucleotides that encodes for the molecules or portions of the chemical entity.

By “linkage” is meant a chemical connecting entity that allows for operatively associating two or more chemical structures, where the linkage is present between the headpiece and one or more tags, between two tags, or between a tag and a tailpiece. The chemical connecting entity can be a non-covalent bond (e.g., as described herein), a covalent bond, or a reaction product between two functional groups. By “chemical linkage” is meant a linkage formed by a non-enzymatic, chemical reaction between two functional groups such as a monophosphate and a hydroxyl group. Exemplary, non-limiting functional groups include a chemical-reactive group, a photo-reactive group, an intercalating moiety, or a cross-linking oligonucleotide (e.g., as described herein). By “enzymatic linkage” is meant an internucleotide or internucleoside linkage formed by an enzyme. Exemplary, non-limiting enzymes include a kinase, a polymerase, a ligase, or combinations thereof. By a linkage “for which a polymerase has reduced ability to read or translocate through” is meant a linkage, when present in an oligonucleotide template that provides a reduced amount of elongated and/or amplified products by a polymerase, as compared to a control oligonucleotide lacking the linkage. Exemplary, non-limiting methods for determining such a linkage include primer extension as assessed by PCR analysis (e.g., quantitative PCR), RT-PCR analysis, liquid chromatography-mass spectrometry, sequence demographics, or other methods. Exemplary, non-limiting polymerases include DNA polymerases and RNA polymerases, such as DNA polymerase I, DNA polymerase II, DNA polymerase III, DNA polymerase VI, Taq DNA polymerase, Deep VentR™ DNA Polymerase (high-fidelity thermophilic DNA polymerase, available from New England Biolabs), T7 DNA polymerase, T4 DNA polymerase, RNA polymerase I, RNA polymerase II, RNA polymerase III, or T7 RNA polymerase.

By “multivalent cation” is meant a cation capable of forming more than one bond with more than one ligand or anion. The multivalent cation can form either an ionic complex or a coordination complex. Exemplary multivalent cations include those from the alkali earth metals (e.g., magnesium) and transition metals (e.g., manganese (II) or cobalt (III)), and those that are optionally bound to one or more anions and/or one or more univalent or polydentate ligands, such as chloride, amine, and/or ethylenediamine.

By “oligonucleotide” is meant a polymer of nucleotides having a 5′-terminus, a 3′-terminus, and one or more nucleotides at the internal position between the 5′- and 3′-termini. The oligonucleotide may include DNA, RNA, or any derivative thereof known in the art that can be synthesized and used for base-pair recognition. The oligonucleotide does not have to have contiguous bases but can be interspersed with linker moieties. The oligonucleotide polymer and nucleotide (e.g., modified DNA or RNA) may include natural bases (e.g., adenosine, thymidine, guanosine, cytidine, uridine, deoxyadenosine, deoxythymidine, deoxyguanosine, deoxycytidine, inosine, or diamino purine), base analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), modified bases (e.g., 2′-substituted nucleotides, such as 2′-O-methylated bases and 2′-fluoro bases), intercalated bases, modified sugars (e.g., 2′-fluororibose; ribose; 2′-deoxyribose; arabinose; hexose; anhydrohexitol; altritol; mannitol; cyclohexanyl; cyclohexenyl; morpholino that also has a phosphoramidate backbone; locked nucleic acids (LNA, e.g., where the 2′-hydroxyl of the ribose is connected by a C₁₋₆ alkylene or C₁₋₆ heteroalkylene bridge to the 4′-carbon of the same ribose sugar, where exemplary bridges included methylene, propylene, ether, or amino bridges); glycol nucleic acid (GNA, e.g., R-GNA or S-GNA, where ribose is replaced by glycol units attached to phosphodiester bonds); threose nucleic acid (TNA, where ribose is replace with α-L-threofuranosyl-(3′→2)); and/or replacement of the oxygen in ribose (e.g., with S, Se, or alkylene, such as methylene or ethylene)), modified backbones (e.g., peptide nucleic acid (PNA), where 2-amino-ethyl-glycine linkages replace the ribose and phosphodiester backbone), and/or modified phosphate groups (e.g., phosphorothioates, 5′-N-phosphoramidites, phosphoroselenates, boranophosphates, boranophosphate esters, hydrogen phosphonates, phosphoramidates, phosphorodiamidates, alkyl or aryl phosphonates, phosphotriesters, bridged phosphoramidates, bridged phosphorothioates, and bridged methylene-phosphonates). The oligonucleotide can be single-stranded (e.g., hairpin), double-stranded, or possess other secondary or tertiary structures (e.g., stem-loop structures, double helixes, triplexes, quadruplexes, etc.). Oligonucleotides may also contain one or more 3′-3′ or 5′-5′ linkages, or one or more inverted nucleotides. This may mean that they contain two 3′-termini or two 5′-termini. Oligonucleotides may also branch one or more times, wherein they may contain more than two termini. Oligonucleotides may also be circularized, wherein they may contain less than two termini and may contain no termini at all.

By “one member of a binding pair” is meant a chemical entity that is capable of forming a pair with another complementary chemical entity for reversible immobilization (e.g., a nucleic acid, a peptide, or a small molecule).

By “operatively linked” or “operatively associated” is meant that two or more chemical structures are directly or indirectly linked together in such a way as to remain linked through the various manipulations they are expected to undergo. Typically, the chemical entity and the headpiece are operatively associated in an indirect manner (e.g., covalently via an appropriate spacer). For example, the spacer may be a bifunctional moiety with a site of attachment for chemical entity and a site of attachment for the headpiece.

By “phosphodiester linkage” is meant a linkage including the structure:

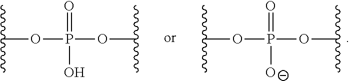 [SMILES: *OP(=O)(O)O* ; *OP(=O)([O-])O*]

By “phosphonate linkage” is meant a linkage including the structure:

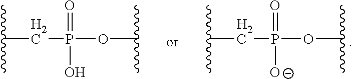 [SMILES: *CP(=O)(O)O* ; *CP(=O)([O-])O*]

By “phosphorothioate linkage” is meant a linkage including the structure:

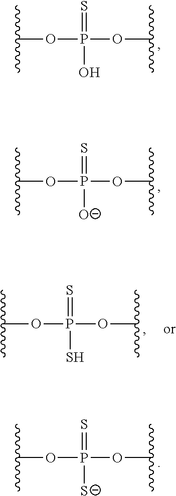 [SMILES: *OP(O)(=S)O* ; *OP([O-])(=S)O* ; *OP(=S)(S)O* ; *OP(=S)([S-])O*]

By “photo-reactive group” is meant a reactive group that participates in a reaction caused by absorption of ultraviolet, visible, or infrared radiation, thus producing a linkage. Exemplary, non-limiting photo-reactive groups are described herein.

By “protecting group” is a meant a group intended to protect the 3′-terminus or 5′-terminus of an oligonucleotide or to protect one or more functional groups of the chemical entity, scaffold, or building block against undesirable reactions during one or more binding steps of making, tagging, or using an oligonucleotide-encoded library. Commonly used protecting groups are disclosed in Greene, “Protective Groups in Organic Synthesis,” 4^{th} Edition (John Wiley & Sons, New York, 2007), which is incorporated herein by reference. Exemplary protecting groups for oligonucleotides include irreversible protecting groups, such as dideoxynucleotides and dideoxynucleosides (ddNTP or ddN), and, more preferably, reversible protecting groups for hydroxyl groups, such as ester groups (e.g., O-(α-methoxyethyl)ester, O-isovaleryl ester, and O-levulinyl ester), trityl groups (e.g., dimethoxytrityl and monomethoxytrityl), xanthenyl groups (e.g., 9-phenylxanthen-9-yl and 9-(p-methoxyphenyl)xanthen-9-yl), acyl groups (e.g., phenoxyacetyl and acetyl), and silyl groups (e.g., t-butyldimethylsilyl). Exemplary, non-limiting protecting groups for chemical entities, scaffolds, and building blocks include N-protecting groups to protect an amino group against undesirable reactions during synthetic procedure (e.g., acyl; aryloyl; carbamyl groups, such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, 4-nitrobenzoyl, and chiral auxiliaries, such as protected or unprotected D, L or D, L-amino acids, such as alanine, leucine, phenylalanine; sulfonyl-containing groups, such as benzenesulfonyl, p-toluenesulfonyl; carbamate forming groups, such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl; alkaryl groups, such as benzyl, triphenylmethyl, benzyloxymethyl; and silyl groups such as trimethylsilyl; where preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz)); O-protecting groups to protect a hydroxyl group against undesirable reactions during synthetic procedure (e.g., alkylcarbonyl groups, such as acyl, acetyl, pivaloyl; optionally substituted arylcarbonyl groups, such as benzoyl; silyl groups, such as trimethylsilyl (TMS), tert-butyldimethylsilyl (TBDMS), tri-iso-propylsilyloxymethyl (TOM), triisopropylsilyl (TIPS); ether-forming groups with the hydroxyl, such methyl, methoxymethyl, tetrahydropyranyl, benzyl, p-methoxybenzyl, trityl; alkoxycarbonyls, such as methoxycarbonyl, ethoxycarbonyl, isopropoxycarbonyl, n-isopropoxycarbonyl, n-butyloxycarbonyl, isobutyloxycarbonyl, sec-butyloxycarbonyl, t-butyloxycarbonyl, 2-ethylhexyloxycarbonyl, cyclohexyloxycarbonyl, methyloxycarbonyl; alkoxyalkoxycarbonyl groups, such as methoxymethoxycarbonyl, ethoxymethoxycarbonyl, 2-methoxyethoxycarbonyl, 2-ethoxyethoxycarbonyl, 2-butoxyethoxycarbonyl, 2-methoxyethoxymethoxycarbonyl, al lyloxycarbonyl, propargyloxycarbonyl, 2-butenoxycarbonyl, 3-methyl-2-butenoxycarbonyl; haloalkoxycarbonyls, such as 2-chloroethoxycarbonyl, 2-chloroethoxycarbonyl, 2,2,2-trichloroethoxycarbonyl; optionally substituted arylalkoxycarbonyl groups, such as benzyloxycarbonyl, p-methylbenzyloxycarbonyl, p-methoxybenzyloxycarbonyl, p-nitrobenzyloxycarbonyl, 2,4-dinitrobenzyloxycarbonyl, 3,5-dimethylbenzyloxycarbonyl, p-chlorobenzyloxycarbonyl, p-bromobenzyloxy-carbonyl; and optionally substituted aryloxycarbonyl groups, such as phenoxycarbonyl, p-nitrophenoxycarbonyl, o-nitrophenoxycarbonyl, 2,4-dinitrophenoxycarbonyl, p-methyl-phenoxycarbonyl, m-methylphenoxycarbonyl, o-bromophenoxycarbonyl, 3,5-dimethylphenoxycarbonyl, p-chlorophenoxycarbonyl, 2-chloro-4-nitrophenoxy-carbonyl); carbonyl-protecting groups (e.g., acetal and ketal groups, such as dimethyl acetal, 1,3-dioxolane; acylal groups; and dithiane groups, such as 1,3-dithianes, 1,3-dithiolane); carboxylic acid-protecting groups (e.g., ester groups, such as methyl ester, benzyl ester, t-butyl ester, orthoesters; silyl groups, such as trimethylsilyl, as well as any described herein; and oxazoline groups); and phosphate-protecting groups (e.g., optionally substituted ester groups, such as methyl ester, isopropyl ester, 2-cyanoethyl ester, allyl ester, t-butyl ester, benzyl ester, fluorenylmethyl ester, 2-(trimethylsilyl)ethyl ester, 2-(methylsulfonyl)ethyl ester, 2,2,2-trichloroethyl ester, 3′,5′-dimethoxybenzoin ester, p-hydroxyphenacyl ester).

By “proximity” or “in proximity” to a terminus of an oligonucleotide is meant near or closer to the stated terminus than the other remaining terminus. For example, a moiety or group in proximity to the 3′-terminus of an oligonucleotide is near or closer to the 3′-terminus than the 5′-terminus. In particular embodiments, a moiety or group in proximity to the 3′-terminus of an oligonucleotide is one, two, three, four, five, six, seven, eight, nine, ten, fifteen, or more nucleotides from the 3′-terminus. In other embodiments, a moiety or group in proximity to the 5′-terminus of an oligonucleotide is one, two, three, four, five, six, seven, eight, nine, ten, fifteen, or more nucleotides from the 5′-terminus.

By “purifying” is meant removing any unreacted product or any agent present in a reaction mixture that may reduce the activity of a chemical or biological agent to be used in a successive step. Purifying can include one or more of chromatographic separation, electrophoretic separation, and precipitation of the unreacted product or reagent to be removed. Purifying may also include the removal of solvent.

By “reversible co-reactive group” is meant a reactive group that participates in a reversible reaction. Exemplary, non-limiting reactive groups include photo-reactive groups, where exposure to a particular absorption radiation results in a linkage between the photo-reactive groups and exposure to a different, particular absorption radiation results in cleavage of the formed linkage (e.g., a cyanovinylcarbazole group, a cyanovinyl group, and an acrylamide group). Another exemplary, non-limiting reactive group includes redox-reactive groups, where such groups can be reversibly reduced or oxidized (e.g., a thiol group).

By “reversible immobilization” is meant immobilization of a complex in a manner which allows for detachment from the support under gentle conditions (e.g., adsorption, ionic binding, affinity binding, chelation, disulfide bond formation, oligonucleotide hybridization, small molecule-small molecule interactions, reversible chemistry, protein-protein interactions, and hydrophobic interactions).

By “scaffold” is meant a chemical moiety that displays one or more diversity nodes in a particular special geometry. Diversity nodes are typically attached to the scaffold during library synthesis, but in some cases one diversity node can be attached to the scaffold prior to library synthesis (e.g., addition of one or more building blocks and/or one or more tags). In some embodiments, the scaffold is derivatized such that it can be orthogonally deprotected during library synthesis and subsequently reacted with different diversity nodes.

By “small molecule” drug or “small molecule” drug candidate is meant a molecule that has a molecular weight below about 1,000 Daltons. Small molecules may be organic or inorganic, isolated (e.g., from compound libraries or natural sources), or obtained by derivatization of known compounds.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, or 20 contiguous amino acids, more preferably at least 25, 50, 75, 90, 100, 150, 200, 250, 300, or 350 contiguous amino acids, and most preferably the full-length amino acid sequence. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

By “substantially” is meant the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

By “tag” or “oligonucleotide tag” is meant an oligonucleotide portion of the library at least part of which encodes information. Non-limiting examples of such information include the addition (e.g., by a binding reaction) of a component (i.e., a scaffold or a building block, as in a scaffold tag or a building block tag, respectively), the headpiece in the library, the identity of the library (i.e., as in an identity tag), the use of the library (i.e., as in a use tag), and/or the origin of a library member (i.e., as in an origin tag). Tag sets may optionally be comprised of equal or nearly-equal mass tags thereby facilitating analytical evaluation of the library by mass spectrometry.

By “tailpiece” is meant an oligonucleotide portion of the library that is attached to the complex after the addition of all of the preceding tags and encodes for the identity of the library, the use of the library, and/or the origin of a library member.

By “primer” is meant an oligonucleotide that is capable of annealing to an oligonucleotide template and then being extended by a polymerase in a template-dependent manner.

Other features and advantages of the invention will be apparent from the following Detailed Description and the claims.

### BRIEF DESCRIPTION OF THE FIGURES

FIG. 1 is an image that illustrates a double-stranded hairpin structure utilized as a headpiece oligonucleotide that offers sites for both chemical ligation of encoding oligonucleotide tags and a protected primary amine for the synthesis of a covalently attached encoded small-molecule.

FIG. 2 is an image of a gel illustrating the progress of an exemplary ligation reaction.

FIG. 3 is an image of two LCMS traces illustrating the progress of an exemplary ligation reaction.

FIG. 4A is an image illustrating a deprotection reaction of a protected amine.

FIG. 4B is an image of a gel illustrating the progress of the deprotection reaction.

FIG. 4C is an image of a LCMS trace illustrating the progress of the deprotection reaction.

FIG. 5A is an image of a mass spectrum of the product of the reaction of HP006 with 1-cyanoimidazole.

FIG. 5B is an image illustrating the reaction of HP006 with 1-cyanoimidazole.

### DETAILED DESCRIPTION

### Encoded Chemical Entities

This invention features methods of producing encoded chemical entities including a chemical entity, one or more tags, and a headpiece operatively associated with the first chemical entity and one or more tags. The chemical entities, headpieces, tags, linkages, and bifunctional spacers are further described below.

### Chemical Entities

The chemical entities or members (e.g., small molecules or peptides) of the invention can include one or more building blocks and optionally include one or more scaffolds.

The scaffold S can be a single atom or a molecular scaffold. Exemplary single atom scaffolds include a carbon atom, a boron atom, a nitrogen atom, or a phosphorus atom, etc. Exemplary polyatomic scaffolds include a cycloalkyl group, a cycloalkenyl group, a heterocycloalkyl group, a heterocycloalkenyl group, an aryl group, or a heteroaryl group. Particular embodiments of a heteroaryl scaffold include a triazine, such as 1,3,5-triazine, 1,2,3-triazine, or 1,2,4-triazine; a pyrimidine; a pyrazine; a pyridazine; a furan; a pyrrole; a pyrrolline; a pyrrolidine; an oxazole; a pyrazole; an isoxazole; a pyran; a pyridine; an indole; an indazole; or a purine.

The scaffold S can be operatively linked to the tag by any useful method. In one example, S is a triazine that is linked directly to the headpiece. To obtain this exemplary scaffold, trichlorotriazine (i.e., a chlorinated precursor of triazine having three chlorines) is reacted with a nucleophilic group of the headpiece. Using this method, S has three positions having chlorine that are available for substitution, where two positions are available diversity nodes and one position is attached to the headpiece. Next, building block Aₙ is added to a diversity node of the scaffold, and tag Aₙ encoding for building block Aₙ (“tag Aₙ”) is ligated to the headpiece, where these two steps can be performed in any order. Then, building block Bₙ is added to the remaining diversity node, and tag Bₙ encoding for building block Bₙ is ligated to the end of tag Aₙ. In another example, S is a triazine that is operatively linked to a tag, where trichlorotriazine is reacted with a nucleophilic group (e.g., an amino group) of a PEG, aliphatic, or aromatic linker of a tag. Building blocks and associated tags can be added, as described above.

In yet another example, S is a triazine that is operatively linked to building block Aₙ. To obtain this scaffold, building block Aₙ having two diversity nodes (e.g., an electrophilic group and a nucleophilic group, such as an Fmoc-amino acid) is reacted with the nucleophilic group of a linker (e.g., the terminal group of a PEG, aliphatic, or aromatic linker, which is attached to a headpiece). Then, trichlorotriazine is reacted with a nucleophilic group of building block Aₙ. Using this method, all three chlorine positions of S are used as diversity nodes for building blocks. As described herein, additional building blocks and tags can be added, and additional scaffolds Sₙ can be added.

Exemplary building block Aₙ's include, e.g., amino acids (e.g., alpha-, beta-, gamma-, delta-, and epsilon-amino acids, as well as derivatives of natural and unnatural amino acids), chemical-reactive reactants (e.g., azide or alkyne chains) with an amine, or a thiol reactant, or combinations thereof. The choice of building block Aₙ depends on, for example, the nature of the reactive group used in the linker, the nature of a scaffold moiety, and the solvent used for the chemical synthesis.

Exemplary building block Bₙ's and Cₙ's include any useful structural unit of a chemical entity, such as optionally substituted aromatic groups (e.g., optionally substituted phenyl or benzyl), optionally substituted heterocyclyl groups (e.g., optionally substituted quinolinyl, isoquinolinyl, indolyl, isoindolyl, azaindolyl, benzimidazolyl, azabenzimidazolyl, benzisoxazolyl, pyridinyl, piperidyl, or pyrrolidinyl), optionally substituted alkyl groups (e.g., optionally substituted linear or branched C₁₋₆ alkyl groups or optionally substituted C₁₋₆ aminoalkyl groups), or optionally substituted carbocyclyl groups (e.g., optionally substituted cyclopropyl, cyclohexyl, or cyclohexenyl). Particularly useful building block Bₙ's and Cₙ's include those with one or more reactive groups, such as an optionally substituted group (e.g., any described herein) having one or optional substituents that are reactive groups or can be chemically modified to form reactive groups. Exemplary reactive groups include one or more of amine (—NR₂, where each R is, independently, H or an optionally substituted C₁₋₆ alkyl), hydroxy, alkoxy (—OR, where R is an optionally substituted C₁₋₆ alkyl, such as methoxy), carboxy (—COOH), amide, or chemical-reactive substituents. A restriction site may be introduced, for example, in tag Bₙ or Cₙ, where a complex can be identified by performing PCR and restriction digest with one of the corresponding restriction enzymes.

### Site for Reversible Immobilization

In some embodiments, the encoded chemical entities optionally include a site for reversible immobilization. Reversible immobilization can be utilized to facilitate buffer-exchange and reagent/contaminant removal during the split-and-mix synthesis of encoded libraries. For example, after a chemical reaction to add a building block to the first chemical entity, the complex may be reversibly immobilized. The excess reagents and solvents may then be removed, the reagents and solvents for the ligation reaction added, and then the complex may be detached from the support. This method incorporates the benefits of solid supported synthesis, such as ease of purification and/or removal of solvents and reagents incompatible with subsequent steps, while allowing the steps that are used to construct the library and oligonucleotide tags to be performed in solution or alternatively while the nascent library is reversibly immobilized.

Exemplary reversible immobilization strategies include: oligonucleotide hybridization including substituted oligonucleotides (2′-modified, PNA, LNA etc.), including double and triple-stranded; Oligonucleotide-ion exchange interactions (e.g. with DEAE-Cellulose); small-molecule-small molecule interactions (e.g. adamantane-cyclodextrin); reversible chemistry (e.g. disulfide bond formation); reversible photochemistry (e.g. cyanovinyl uridine photo-cross-linking); reversible chemical cross-linking (e.g. with an exogenously added reactive entity); immobilized metal affinity chromatography (e.g., immobilized Ni-NTA with His₆); antibody-epitope interaction (e.g. immobilized anti-FLAG antibody and FLAG peptide); protein-protein interaction; protein-small-molecule interaction (e.g. immobilized streptavidin with iminobiotin or immobilized maltose-binding protein and maltose); reversible oligonucleotide ligation (e.g. the ligation of restricted dsDNA followed by restriction); and hydrophobic interaction (e.g. a fluorous tag and a hydrophobic surface). In some embodiments, the site for reversible immobilization comprises one member of a binding pair of any of the reversible immobilization strategies described herein, e.g., a nucleic acid, peptide, or small molecule.

Headpiece

In an encoded chemical entity, the headpiece operatively links each chemical entity to its encoding oligonucleotide tag. Generally, the headpiece is a starting oligonucleotide having at least two functional groups that can be further derivatized, where the first functional group operatively links the first chemical entity (or a component thereof) to the headpiece and the second functional group operatively links one or more tags to the headpiece. A bifunctional spacer can optionally be used as a spacing moiety between the headpiece and a chemical entity.

The functional groups of the headpiece can be used to form a covalent bond with a component of a chemical entity and another covalent bond with a tag. The component can be any part of the small molecule, such as a scaffold having diversity nodes or a building block. Alternatively, the headpiece can be derivatized to provide a spacer (e.g., a spacing moiety separating the headpiece from the small molecule to be formed in the library) terminating in a functional group (e.g., a hydroxyl, amine, carboxyl, sulfhydryl, alkynyl, azido, or phosphate group), which is used to form the covalent linkage with a component of the chemical entity. The spacer can be attached to the 5′-terminus, at one of the internal positions, or to the 3′-terminus of the headpiece. When the spacer is attached to one of the internal positions, the spacer can be operatively linked to a derivatized base (e.g., the C5 position of uridine) or placed internally within the oligonucleotide using standard techniques known in the art. Exemplary spacers are described herein.

The headpiece can have any useful structure. The headpiece can be, e.g., 1 to 100 nucleotides in length, preferably 5 to 20 nucleotides in length, and most preferably 5 to 15 nucleotides in length. The headpiece can be single-stranded or double-stranded and can consist of natural or modified nucleotides, as described herein. For example, the chemical moiety can be operatively linked to the 3′-terminus or 5′-terminus of the headpiece. In particular embodiments, the headpiece includes a hairpin structure formed by complementary bases within the sequence. For example, the chemical moiety can be operatively linked to the internal position, the 3′-terminus, or the 5′-terminus of the headpiece.

Generally, the headpiece includes a non-self-complementary sequence on the 5′- or 3′-terminus that allows for binding an oligonucleotide tag by polymerization, enzymatic ligation, or chemical reaction. The headpiece can allow for ligation of oligonucleotide tags and optional purification and phosphorylation steps. After the addition of the last tag, an additional adapter sequence can be added to the 5′-terminus of the last tag. Exemplary adapter sequences include a primer-binding sequence or a sequence having a label (e.g., biotin). In cases where many building blocks and corresponding tags are used (e.g., 100), a mix-and-split strategy may be employed during the oligonucleotide synthesis step to create the necessary number of tags. Such mix-and-split strategies for DNA synthesis are known in the art. The resultant library members can be amplified by PCR following selection for binding entities versus a target(s) of interest.

The headpiece or the complex can optionally include one or more primer-binding sequences. For example, the headpiece has a sequence in the loop region of the hairpin that serves as a primer-binding region for amplification, where the primer-binding region has a higher melting temperature for its complementary primer (e.g., which can include flanking identifier regions) than for a sequence in the headpiece. In other embodiments, the complex includes two primer-binding sequences (e.g., to enable a PCR reaction) on either side of one or more tags that encode one or more building blocks. Alternatively, the headpiece may contain one primer-binding sequence on the 5′- or 3′-terminus. In other embodiments, the headpiece is a hairpin, and the loop region forms a primer-binding site or the primer-binding site is introduced through hybridization of an oligonucleotide to the headpiece on the 3′ side of the loop. A primer oligonucleotide, containing a region homologous to the 3′-terminus of the headpiece and carrying a primer-binding region on its 5′-terminus (e.g., to enable a PCR reaction) may be hybridized to the headpiece and may contain a tag that encodes a building block or the addition of a building block. The primer oligonucleotide may contain additional information, such as a region of randomized nucleotides, e.g., 2 to 16 nucleotides in length, which is included for bioinformatics analysis.

The headpiece can optionally include a hairpin structure, where this structure can be achieved by any useful method. For example, the headpiece can include complementary bases that form intermolecular base pairing partners, such as by Watson-Crick DNA base pairing (e.g., adenine-thymine and guanine-cytosine) and/or by wobble base pairing (e.g., guanine-uracil, inosine-uracil, inosine-adenine, and inosine-cytosine). In another example, the headpiece can include modified or substituted nucleotides that can form higher affinity duplex formations compared to unmodified nucleotides, such modified or substituted nucleotides being known in the art. In yet another example, the headpiece includes one or more cross-linked bases to form the hairpin structure. For example, bases within a single strand or bases in different double strands can be cross-linked, e.g., by using psoralen.

The headpiece or complex can optionally include one or more labels that allow for detection. For example, the headpiece, one or more oligonucleotide tags, and/or one or more primer sequences can include an isotope, a radioimaging agent, a marker, a tracer, a fluorescent label (e.g., rhodamine or fluorescein), a chemiluminescent label, a quantum dot, and a reporter molecule (e.g., biotin or a his-tag).

In other embodiments, the headpiece or tag may be modified to support solubility in semi-, reduced-, or non-aqueous (e.g., organic) conditions. Nucleotide bases of the headpiece or tag can be rendered more hydrophobic by modifying, for example, the C5 positions of T or C bases with aliphatic chains without significantly disrupting their ability to hydrogen bond to their complementary bases. Exemplary modified or substituted nucleotides are 5′-dimethoxytrityl-N4-diisobutylaminomethylidene-5-(1-propynyl)-2′-deoxycytidine,3′-[(2-cyanoethyl)-(N,N-diisopropyl)]-phosphoramidite; 5′-dimethoxytrityl-5-(1-propynyl)-2′-deoxyuridine,3′-[(2-cyanoethyl)-(N,N-diisopropyl)]-phosphoramidite; 5′-dimethoxytrityl-5-fluoro-2′-deoxyuridine,3′-[(2-cyanoethyl)-(N,N-diisopropyl)]-phosphoramidite; and 5′-dimethoxytrityl-5-(pyren-1-yl-ethynyl)-2′-deoxyuridine, or 3′-[(2-cyanoethyl)-(N,N-diisopropyl)]-phosphoramidite.

In addition, the headpiece oligonucleotide can be interspersed with modifications that promote solubility in organic solvents. For example, azobenzene phosphoramidite can introduce a hydrophobic moiety into the headpiece design. Such insertions of hydrophobic amidites into the headpiece can occur anywhere in the molecule. However, the insertion cannot interfere with subsequent tagging using additional DNA tags during the library synthesis or ensuing PCR once a selection is complete or microarray analysis, if used for tag deconvolution. Such additions to the headpiece design described herein would render the headpiece soluble in, for example, 15%, 25%, 30%, 50%, 75%, 90%, 95%, 98%, 99%, or 100% organic solvent. Thus, addition of hydrophobic residues into the headpiece design allows for improved solubility in semi- or non-aqueous (e.g., organic) conditions, while rendering the headpiece competent for oligonucleotide tagging. Furthermore, DNA tags that are subsequently introduced into the library can also be modified at the C5 position of T or C bases such that they also render the library more hydrophobic and soluble in organic solvents for subsequent steps of library synthesis.

In particular embodiments, the headpiece and the first tag can be the same entity, i.e., a plurality of headpiece-tag entities can be constructed that all share common parts (e.g., a primer-binding region) and all differ in another part (e.g., encoding region). These may be utilized in the “split” step and pooled after the event they are encoding has occurred.

In particular embodiments, the headpiece can encode information, e.g., by including a sequence that encodes the first split(s) step or a sequence that encodes the identity of the library, such as by using a particular sequence related to a specific library.

Oligonucleotide Tags

The oligonucleotide tags described herein (e.g., a tag or a portion of a headpiece or a portion of a tailpiece) can be used to encode any useful information, such as a molecule, a portion of a chemical entity, the addition of a component (e.g., a scaffold or a building block), a headpiece in the library, the identity of the library, the use of one or more library members (e.g., use of the members in an aliquot of a library), and/or the origin of a library member (e.g., by use of an origin sequence).

Any sequence in an oligonucleotide can be used to encode any information. Thus, one oligonucleotide sequence can serve more than one purpose, such as to encode two or more types of information or to provide a starting oligonucleotide that also encodes for one or more types of information. For example, the first tag can encode for the addition of a first building block, as well as for the identification of the library. In another example, a headpiece can be used to provide a starting oligonucleotide that operatively links a chemical entity to a tag, where the headpiece additionally includes a sequence that encodes for the identity of the library (i.e., the library-identifying sequence). Accordingly, any of the information described herein can be encoded in separate oligonucleotide tags or can be combined and encoded in the same oligonucleotide sequence (e.g., an oligonucleotide tag, such as a tag, or a headpiece).

A building block sequence encodes for the identity of a building block and/or the type of binding reaction conducted with a building block. This building block sequence is included in a tag, where the tag can optionally include one or more types of sequence described below (e.g., a library-identifying sequence, a use sequence, and/or an origin sequence).

A library-identifying sequence encodes for the identity of a particular library. In order to permit mixing of two or more libraries, a library member may contain one or more library-identifying sequences, such as in a library-identifying tag (i.e., an oligonucleotide including a library-identifying sequence), in a ligated tag, in a part of the headpiece sequence, or in a tailpiece sequence. These library-identifying sequences can be used to deduce encoding relationships, where the sequence of the tag is translated and correlated with chemical (synthesis) history information. Accordingly, these library-identifying sequences permit the mixing of two or more libraries together for selection, amplification, purification, sequencing, etc.

A use sequence encodes the history (i.e., use) of one or more library members in an individual aliquot of a library. For example, separate aliquots may be treated with different reaction conditions, building blocks, and/or selection steps. In particular, this sequence may be used to identify such aliquots and deduce their history (use) and thereby permit the mixing together of aliquots of the same library with different histories (uses) (e.g., distinct selection experiments) for the purposes of the mixing together of samples together for selection, amplification, purification, sequencing, etc. These use sequences can be included in a headpiece, a tailpiece, a tag, a use tag (i.e., an oligonucleotide including a use sequence), or any other tag described herein (e.g., a library-identifying tag or an origin tag).

An origin sequence is a degenerate (random, stochastically-generated) oligonucleotide sequence of any useful length (e.g., about six oligonucleotides) that encodes for the origin of the library member. This sequence serves to stochastically subdivide library members that are otherwise identical in all respects into entities distinguishable by sequence information, such that observations of amplification products derived from unique progenitor templates (e.g., selected library members) can be distinguished from observations of multiple amplification products derived from the same progenitor template (e.g., a selected library member). For example, after library formation and prior to the selection step, each library member can include a different origin sequence, such as in an origin tag. After selection, selected library members can be amplified to produce amplification products, and the portion of the library member expected to include the origin sequence (e.g., in the origin tag) can be observed and compared with the origin sequence in each of the other library members. As the origin sequences are degenerate, each amplification product of each library member should have a different origin sequence. However, an observation of the same origin sequence in the amplification product could indicate multiple amplicons derived from the same template molecule. When it is desired to determine the statistics and demographics of the population of encoding tags prior to amplification, as opposed to post-amplification, the origin tag may be used. These origin sequences can be included in a headpiece, a tailpiece, a tag, an origin tag (i.e., an oligonucleotide including an origin sequence), or any other tag described herein (e.g., a library-identifying tag or a use tag).

Any of the types of sequences described herein can be included in the headpiece. For example, the headpiece can include one or more of a building block sequence, a library-identifying sequence, a use sequence, or an origin sequence.

Any of these sequences described herein can be included in a tailpiece. For example, the tailpiece can include one or more of a library-identifying sequence, a use sequence, or an origin sequence.

Any of tags described herein can include a connector at or in proximity to the 5′- or 3′-terminus having a fixed sequence. Connectors facilitate the formation of linkages (e.g., chemical linkages) by providing a reactive group (e.g., a chemical-reactive group or a photo-reactive group) or by providing a site for an agent that allows for a linkage (e.g., an agent of an intercalating moiety or a reversible reactive group in the connector(s) or cross-linking oligonucleotide). Each 5′-connector may be the same or different, and each 3′-connector may be the same or different. In an exemplary, non-limiting complex having more than one tags, each tag can include a 5′-connector and a 3′-connector, where each 5′-connector has the same sequence and each 3′-connector has the same sequence (e.g., where the sequence of the 5′-connector can be the same or different from the sequence of the 3′-connector). The connector provides a sequence that can be used for one or more linkages. To allow for binding of a relay primer or for hybridizing a cross-linking oligonucleotide, the connector can include one or more functional groups allowing for a linkage (e.g., a linkage for which a polymerase has reduced ability to read or translocate through, such as a chemical linkage).

These sequences can include any modification described herein for oligonucleotides, such as one or more modifications that promote solubility in organic solvents (e.g., any described herein, such as for the headpiece), that provide an analog of the natural phosphodiester linkage (e.g., a phosphorothioate analog), or that provide one or more non-natural oligonucleotides (e.g., 2′-substituted nucleotides, such as 2′-O-methylated nucleotides and 2′-fluoro nucleotides, or any described herein).

These sequences can include any characteristics described herein for oligonucleotides. For example, these sequences can be included in tag that is less than 20 nucleotides (e.g., as described herein). In other examples, the tags including one or more of these sequences have about the same mass (e.g., each tag has a mass that is about +/−10% from the average mass between within a specific set of tags that encode a specific variable); lack a primer-binding (e.g., constant) region; lack a constant region; or have a constant region of reduced length (e.g., a length less than 30 nucleotides, less than 25 nucleotides, less than 20 nucleotides, less than 19 nucleotides, less than 18 nucleotides, less than 17 nucleotides, less than 16 nucleotides, less than 15 nucleotides, less than 14 nucleotides, less than 13 nucleotides, less than 12 nucleotides, less than 11 nucleotides, less than 10 nucleotides, less than 9 nucleotides, less than 8 nucleotides, or less than 7 nucleotides).

Sequencing strategies for libraries and oligonucleotides of this length may optionally include concatenation or catenation strategies to increase read fidelity or sequencing depth, respectively. In particular, the selection of encoded libraries that lack primer-binding regions has been described in the literature for SELEX, such as described in Jarosch et al., Nucleic Acids Res. 34: e86 (2006), which is incorporated herein by reference. For example, a library member can be modified (e.g., after a selection step) to include a first adapter sequence on the 5′-terminus of the complex and a second adapter sequence on the 3′-terminus of the complex, where the first sequence is substantially complementary to the second sequence and result in forming a duplex. To further improve yield, two fixed dangling nucleotides (e.g., CC) are added to the 5′-terminus.

Linkages

The linkages of the invention are present between oligonucleotides that encode information (e.g., such as between the headpiece and a tag, between two tags, or between a tag and a tailpiece). Exemplary linkages include phosphodiesters, phosphonates, and phosphorothioates. In some embodiments, a polymerase has reduced ability to read or translocate through one or more linkages. In certain embodiments, chemical linkages include one or more of a chemical-reactive group such as a monophosphate and/or a hydroxyl group, a photo-reactive group, an intercalating moiety, a cross-linking oligonucleotide, or a reversible co-reactive group.

A linkage may be tested to determine whether a polymerase has reduced ability to read or translocate through that linkage. This ability can be tested by any useful method, such as liquid chromatography-mass spectrometry, RT-PCR analysis, sequence demographics, and/or PCR analysis.

In some embodiments, chemical ligation includes the use of one or more chemical-reactive pairs to provide a linkage such as a monophosphate and a hydroxyl. As described herein, readable linkages may be synthesized by chemical ligation, for example, by reaction of a monophosphate, a monophosphotioate, or monophosphanate on a 5′- or 3′-terminus with a hydroxyl group on a 5′- or 3′-terminus in the presence of cyanoimidazole and a divalent metal source (e.g., ZnCl₂).

Other exemplary chemical-reactive pairs are a pair including an optionally substituted alkynyl group and an optionally substituted azido group to form a triazole via a Huisgen 1,3-dipolar cycloaddition reaction; an optionally substituted diene having a 4π-electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and an optionally substituted dienophile or an optionally substituted heterodienophile having a 2π-electron system (e.g., an optionally substituted alkenyl group or an optionally substituted alkynyl group) to form a cycloalkenyl via a Diels-Alder reaction; a nucleophile (e.g., an optionally substituted amine or an optionally substituted thiol) with a strained heterocyclyl electrophile (e.g., optionally substituted epoxide, aziridine, aziridinium ion, or episulfonium ion) to form a heteroalkyl via a ring opening reaction; a phosphorothioate group with an iodo group, such as in a splinted ligation of an oligonucleotide containing 5′-iodo dT with a 3′-phosphorothioate oligonucleotide; an optionally substituted amino group with an aldehyde group or a ketone group, such as a reaction of a 3′-aldehyde-modified oligonucleotide, which can optionally be obtained by oxidizing a commercially available 3′-glyceryl-modified oligonucleotide, with 5′-amino oligonucleotide (i.e., in a reductive amination reaction) or a 5′-hydrazido oligonucleotide; a pair of an optionally substituted amino group and a carboxylic acid group or a thiol group (e.g., with or without the use of succinimidyl trans-4-(maleimidylmethyl)cyclohexane-1-carboxylate (SMCC) or 1-ethyl-3-(3-dimethylaminopropyl)carbodiimide (EDAC); a pair of an optionally substituted hydrazine and an aldehyde or a ketone group; a pair of an optionally substituted hydroxylamine and an aldehyde or a ketone group; or a pair of a nucleophile and an optionally substituted alkyl halide.

Platinum complexes, alkylating agents, or furan-modified nucleotides can also be used as a chemical-reactive group to form inter- or intra-strand linkages. Such agents can be used between two oligonucleotides and can optionally be present in the cross-linking oligonucleotide.

Exemplary, non-limiting platinum complexes include cisplatin (cis-diamminedichloroplatinum (II), e.g., to form GG intra-strand linkages), transplatin (trans-diaminedichloroplatinum (II), e.g., to form GXG inter-strand linkages, where X can be any nucleotide), carboplatin, picolatin (ZD0473), ormaplatin, or oxaliplatin to form, e.g., GC, CG, AG, or GG linkages. Any of these linkages can be inter- or intra-strand linkages.

Exemplary, non-limiting alkylating agents include nitrogen mustard (mechlorethamine, e.g., to form GG linkages), chlorambucil, melphalan, cyclophosphamide, prodrug forms of cyclophosphamide (e.g., 4-hydroperoxycyclophosphamide and ifosfamide)), 1,3-bis(2-chloroethyl)-1-nitrosourea (BCNU, carmustine), an aziridine (e.g., mitomycin C, triethylenemelamine, or triethylenethiophosphoramide (thio-tepa) to form GG or AG linkages), hexamethylmelamine, an alkyl sulfonate (e.g., busulphan to form GG linkages), or a nitrosourea (e.g., 2-chloroethylnitrosourea to form GG or CG linkages, such as carmustine (BCNU), chlorozotocin, lomustine (CCNU), and semustine (methyl-CCNU)). Any of these linkages can be inter- or intra-strand linkages.

Furan-modified nucleotides can also be used to form linkages. Upon in situ oxidation (e.g., with N-bromosuccinimide (NBS)), the furan moiety forms a reactive oxo-enal derivative that reacts with a complementary base to form an inter-strand linkage. In some embodiments, the furan-modified nucleotides forms linkages with a complementary A or C nucleotide. Exemplary, non-limiting furan-modified nucleotides include any 2′-(furan-2-yl)propanoylamino-modified nucleotide; or an acyclic, modified nucleotides of 2-(furan-2-yl)ethyl glycol nucleic acid.

Photo-reactive groups can also be used as a reactive group. Exemplary, non-limiting photo-reactive groups include an intercalating moiety, a psoralen derivative (e.g., psoralen, HMT-psoralen, or 8-methoxypsoralen), an optionally substituted cyanovinylcarbazole group, an optionally substituted vinylcarbazole group, an optionally substituted cyanovinyl group, an optionally substituted acrylamide group, an optionally substituted diazirine group, an optionally substituted benzophenone (e.g., succinimidyl ester of 4-benzoylbenzoic acid or benzophenone isothiocyanate), an optionally substituted 5-(carboxy)vinyl-uridine group (e.g., 5-(carboxy)vinyl-2′-deoxyuridine), or an optionally substituted azide group (e.g., an aryl azide or a halogenated aryl azide, such as succinimidyl ester of 4-azido-2,3,5,6-tetrafluorobenzoic acid (ATFB)).

Intercalating moieties can also be used as a reactive group. Exemplary, non-limiting intercalating moieties include a psoralen derivative, an alkaloid derivative (e.g., berberine, palmatine, coralyne, sanguinarine (e.g., iminium or alkanolamine forms thereof), or aristololactam-β-D-glucoside), an ethidium cation (e.g., ethidium bromide), an acridine derivative (e.g., proflavine, acriflavine, or amsacrine), an anthracycline derivative (e.g., doxorubicin, epirubicin, daunorubicin (daunomycin), idarubicin, and aclarubicin), or thalidomide.

For a cross-linking oligonucleotide, any useful reactive group (e.g., described herein) can be used to form inter- or intra-strand linkages. Exemplary reactive groups include chemical-reactive group, a photo-reactive group, an intercalating moiety, and a reversible co-reactive group. Cross-linking agents for use with cross-linking oligonucleotides include, without limitation, alkylating agents (e.g., as described herein), cisplatin (cis-diamminedichloroplatinum(II)), trans-diaminedichloroplatinum(II), psoralen, HMT-psoralen, 8-methoxypsoralen, furan-modified nucleotides, 2-fluoro-deoxyinosine (2-F-dI), 5-bromo-deoxycytosine (5-Br-dC), 5-bromo deoxyuridine (5-Br-dU), 5-iodo-deoxycytosine (5-I-dC), 5-iodo-deoxyuridine (5-I-dU), succinimidyl trans-4-(maleimidylmethyl)cyclohexane-1-carboxylate, SMCC, EDAC, or succinimidyl acetylthioacetate (SATA).

Oligonucleotides can also be modified to contain thiol moieties that can be reacted with a variety of thiol reactive groups such as maleimides, halogens, and iodoacetamides and thus can be used for cross-linking two oligonucleotides. The thiol groups can be linked to the 5′- or the 3′-terminus of an oligonucleotide.

For inter-strand cross-linking between duplex oligonucleotides at a pyrimidine (e.g., thymidine) position, the intercalating, photo-reactive moiety psoralen can be chosen. Psoralen intercalates into the duplex and forms covalent inter-strand cross-links with pyrimidines, preferentially at 5′-TpA sites, upon irradiation with ultraviolet light (about 254 nm). The psoralen moiety can be covalently attached to a modified oligonucleotide (e.g., by an alkane chain, such as a C₁₋₁₀ alkyl, or a polyethylene glycol group, such as —(CH₂CH₂O)ₙCH₂CH₂—, where n is an integer from 1 to 50). Exemplary psoralen derivatives can also be used, where non-limiting derivatives include 4′-(hydroxyethoxymethy)-4,5′,8-trimethylpsoralen (HMT-psoralen) and 8-methoxypsoralen.

Various portions of the cross-linking oligonucleotide can be modified to introduce a linkage. For example, terminal phosphorothioates in oligonucleotides can also be used for linking two adjacent oligonucleotides. Halogenated uracils/cytosines can also be used as cross-linker modifications in the oligonucleotide. For example, 2-fluoro-deoxyinosine (2-F-dl) modified oligonucleotides can be reacted with disulfide-containing diamines or thiopropylamines to form disulfide linkages.

As described below, reversible co-reactive groups include those selected from a cyanovinylcarbazole group, a cyanovinyl group, an acrylamide group, a thiol group, or a sulfonylethyl thioethers. An optionally substituted cyanovinylcarbazole (CNV) group can also be used in oligonucleotides to cross-link to a pyrimidine base (e.g., cytosine, thymine, and uracil, as well as modified bases thereof) in complementary strands. CNV groups promote [2+2] cycloaddition with the adjacent pyrimidine base upon irradiation at 366 nm, which results in an inter-strand cross-link. Irradiation at 312 nm reverses the cross-link and thus provides a method for reversible cross-linking of oligonucleotide strands. A non-limiting CNV group is 3-cyanovinylcarbozaole, which can be included as a carboxyvinylcarbazole nucleotide (e.g., as 3-carboxyvinylcarbazole-1′-β-deoxyriboside-5′-triphosphate).

The CNV group can be modified to replace the reactive cyano group with another reactive group to provide an optionally substituted vinylcarbazole group. Exemplary non-limiting reactive groups for a vinylcarbazole group include an amide group of —CONR_{N1}R_{N2}, where each R_{N1} and R_{N2} can be the same or different and is independently H and C₁₋₆ alkyl, e.g., —CONH₂; a carboxyl group of —CO₂H; or a C₂₋₇ alkoxycarbonyl group (e.g., methoxycarbonyl). Furthermore, the reactive group can be located on the alpha or beta carbon of the vinyl group. Exemplary vinylcarbazole groups include a cyanovinylcarbazole group, as described herein; an amidovinylcarbazole group (e.g., an amidovinylcarbazole nucleotide, such as 3-amidovinylcarbazole-1′-β-deoxyriboside-5′-triphosphate); a carboxyvinylcarbazole group (e.g., a carboxyvinylcarbazole nucleotide, such as 3-carboxyvinylcarbazole-1′-β-deoxyriboside-5′-triphosphate); and a C₂₋₇ alkoxycarbonylvinylcarbazole group (e.g., an alkoxycarbonylvinylcarbazole nucleotide, such as 3-methoxycarbonylvinylcarbazole-1′-β-deoxyriboside-5′-triphosphate). Additional optionally substituted vinylcarbazole groups and nucleotides having such groups are provided in the chemical formulas of U.S. Pat. No. 7,972,792 and Yoshimura and Fujimoto, Org. Lett. 10:3227-3230 (2008), which are both hereby incorporated by reference in their entirety.

Other reversible reactive groups include a thiol group and another thiol group to form a disulfide, as well as a thiol group and a vinyl sulfone group to form a sulfonylethyl thioethers. Thiol-thiol groups can optionally include a linkage formed by a reaction with bis-((N-iodoacetyl)piperazinyl)sulfonerhodamine. Other reversible reactive groups (e.g., such as some photo-reactive groups) include optionally substituted benzophenone groups. A non-limiting example is benzophenone uracil (BPU), which can be used for site- and sequence-selective formation of an interstrand cross-link of BPU-containing oligonucleotide duplexes. This cross-link can be reversed upon heating, providing a method for the reversible cross-linking of two oligonucleotide strands.

In other embodiments, chemical ligation includes introducing an analog of the phosphodiester bond, e.g., for post-selection PCR analysis and sequencing. Exemplary analogs of a phosphodiester include a phosphorothioate linkage (e.g., as introduced by use of a phosphorothioate group and a leaving group, such as an iodo group), a phosphoramide linkage, or a phosphorodithioate linkage (e.g., as introduced by use of a phosphorodithioate group and a leaving group, such as an iodo group). For any of the groups described herein (e.g., a chemical-reactive group, a photo-reactive group, an intercalating moiety, a cross-linking oligonucleotide, or a reversible co-reactive group), the group can be incorporated at or in proximity to the terminus of an oligonucleotide or between the 5′- and 3′-termini. Furthermore, one or more groups can be present in each oligonucleotide. When pairs of reactive groups are required, then oligonucleotides can be designed to facilitate a reaction between the pair of groups. In the non-limiting example of a cyanovinylcarbazole group that co-reacts with a pyrimidine base, the first oligonucleotide can be designed to include the cyanovinylcarbazole group at or in proximity to the 5′-terminus. In this example, a second oligonucleotide can be designed to be complementary to the first oligonucleotide and to include the co-reactive pyrimidine base at a position that aligns with the cyanovinylcarbazole group when the first and second oligonucleotide hybridizes. Any of the groups herein and any of the oligonucleotides having one or more groups can be designed to facilitate reaction between the groups to form one or more linkages.

Bifunctional Spacers

The bifunctional spacer between the headpiece and a chemical entity can be varied to provide an appropriate spacing moiety and/or to increase the solubility of the headpiece in organic solvent. A wide variety of spacers are commercially available that can couple the headpiece with the small molecule library. The spacer typically consists of linear or branched chains and may include a C₁₋₁₀ alkyl, a heteroalkyl of 1 to 10 atoms, a C₂₋₁₀ alkenyl, a C₂₋₁₀ alkynyl, C₅₋₁₀ aryl, a cyclic or polycyclic system of 3 to 20 atoms, a phosphodiester, a peptide, an oligosaccharide, an oligonucleotide, an oligomer, a polymer, or a poly alkyl glycol (e.g., a poly ethylene glycol, such as —(CH₂CH₂O)ₙCH₂CH₂—, where n is an integer from 1 to 50), or combinations thereof.

The bifunctional spacer may provide an appropriate spacing moiety between the headpiece and a chemical entity of the library. In certain embodiments, the bifunctional spacer includes three parts. Part 1 may be a reactive group, which forms a covalent bond with DNA, such as, e.g., a carboxylic acid, preferably activated by a N-hydroxy succinimide (NHS) ester to react with an amino group on the DNA (e.g., amino-modified dT), an amidite to modify the 5′ or 3′-terminus of a single-stranded headpiece (achieved by means of standard oligonucleotide chemistry), chemical-reactive pairs (e.g., azido-alkyne cycloaddition in the presence of Cu(I) catalyst, or any described herein), or thiol reactive groups. Part 2 may also be a reactive group, which forms a covalent bond with the chemical entity, either building block Aₙ or a scaffold. Such a reactive group could be, e.g., an amine, a thiol, an azide, or an alkyne. Part 3 may be a chemically inert spacing moiety of variable length, introduced between Part 1 and 2. Such a spacing moiety can be a chain of ethylene glycol units (e.g., PEGs of different lengths), an alkane, an alkene, a polyene chain, or a peptide chain. The spacer can contain branches or inserts with hydrophobic moieties (such as, e.g., benzene rings) to improve solubility of the headpiece in organic solvents, as well as fluorescent moieties (e.g. fluorescein or Cy-3) used for library detection purposes. Hydrophobic residues in the headpiece design may be varied with the spacer design to facilitate library synthesis in organic solvents. For example, the headpiece and spacer combination is designed to have appropriate residues wherein the octanol:water coefficient (P_{oct}) is from, e.g., 1.0 to 2.5.

Spacers can be empirically selected for a given small molecule library design, such that the library can be synthesized in organic solvent, for example, in 15%, 25%, 30%, 50%, 75%, 90%, 95%, 98%, 99%, or 100% organic solvent. The spacer can be varied using model reactions prior to library synthesis to select the appropriate chain length that solubilizes the headpiece in an organic solvent. Exemplary spacers include those having increased alkyl chain length, increased poly ethylene glycol units, branched species with positive charges (to neutralize the negative phosphate charges on the headpiece), or increased amounts of hydrophobicity (for example, addition of benzene ring structures).

Examples of commercially available spacers include amino-carboxylic spacers, such as those being peptides (e.g., Z-Gly-Gly-Gly-Osu (N-alpha-benzyloxycarbonyl-(Glycine)₃-N-succinimidyl ester) or Z-Gly-Gly-Gly-Gly-Gly-Gly-Osu (N-alpha-benzyloxycarbonyl-(Glycine)₆-N-succinimidyl ester, SEQ ID NO: 1)), PEG (e.g., Fmoc-aminoPEG2000-NHS or amino-PEG (12-24)-NHS), or alkane acid chains (e.g., Boc-ε-aminocaproic acid-Osu); chemical-reactive pair spacers, such as those chemical-reactive pairs described herein in combination with a peptide moiety (e.g., azidohomoalanine-Gly-Gly-Gly-OSu (SEQ ID NO: 2) or propargylglycine-Gly-Gly-Gly-OSu (SEQ ID NO: 3)), PEG (e.g., azido-PEG-NHS), or an alkane acid chain moiety (e.g., 5-azidopentanoic acid, (S)-2-(azidomethyl)-1-Boc-pyrrolidine, 4-azidoaniline, or 4-azido-butan-1-oic acid N-hydroxysuccinimide ester); thiol-reactive spacers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]-propionamido)hexanoate)); and amidites for oligonucleotide synthesis, such as amino modifiers (e.g., 6-(trifluoroacetylamino)-hexyl-(2-cyanoethyl)-(N,N-diisopropyl)-phosphoramidite), thiol modifiers (e.g., S-trityl-6-mercaptohexyl-1-[(2-cyanoethyl)-(N,N-diisopropyl)]-phosphoramidite, or chemical-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-1-O-succinoyl, long chain alkylamino CPG, or 4-azido-butan-1-oic acid N-hydroxysuccinimide ester)). Additional spacers are known in the art, and those that can be used during library synthesis include, but are not limited to, 5′-O-dimethoxytrityl-1′,2′-dideoxyribose-3′-[(2-cyanoethyl)-(N,N-diisopropyl)]-phosphoramidite; 9-O-dimethoxytrityl-triethylene glycol,1-[(2-cyanoethyl)-(N,N-diisopropyl)]-phosphoramidite; 3-(4,4′-dimethoxytrityloxy)propyl-1-[(2-cyanoethyl)-(N,N-diisopropyl)]-phosphoramidite; and 18-O-dimethoxytrityl hexaethyleneglycol,1-[(2-cyanoethyl)-(N,N-diisopropyl)]-phosphoramidite. Any of the spacers herein can be added in tandem to one another in different combinations to generate spacers of different desired lengths.

Spacers may also be branched, where branched spacers are well known in the art and examples can consist of symmetric or asymmetric doublers or a symmetric trebler. See, for example, Newcome et al., Dendritic Molecules: Concepts, Synthesis, Perspectives, VCH Publishers (1996); Boussif et al., Proc. Natl. Acad. Sci. USA 92:7297-7301 (1995); and Jansen et al., Science 266:1226 (1994).

Enzymatic Ligation and Chemical Ligation Techniques

Various ligation techniques can be used to add tags, to the headpiece to produce a complex. Accordingly, any of the binding steps described herein can include any useful ligation techniques, such as enzymatic ligation and/or chemical ligation. These binding steps can include the addition of one or more tags to the headpiece or complex. In particular embodiments, the ligation techniques used for any oligonucleotide provide a resultant product that can be transcribed and/or reverse transcribed to allow for decoding of the library or for template-dependent polymerization with one or more DNA or RNA polymerases.

Generally, enzymatic ligation produces an oligonucleotide having a native phosphodiester bond that can be transcribed and/or reverse transcribed. Exemplary methods of enzyme ligation are provided herein and include the use of one or more RNA or DNA ligases, such as T4 RNA ligase 1 or 2, T4 DNA ligase, CircLigase™ ssDNA ligase, CircLigase™ II ssDNA ligase, and ThermoPhage™ ssDNA ligase (Prokazyme Ltd., Reykjavik, Iceland).

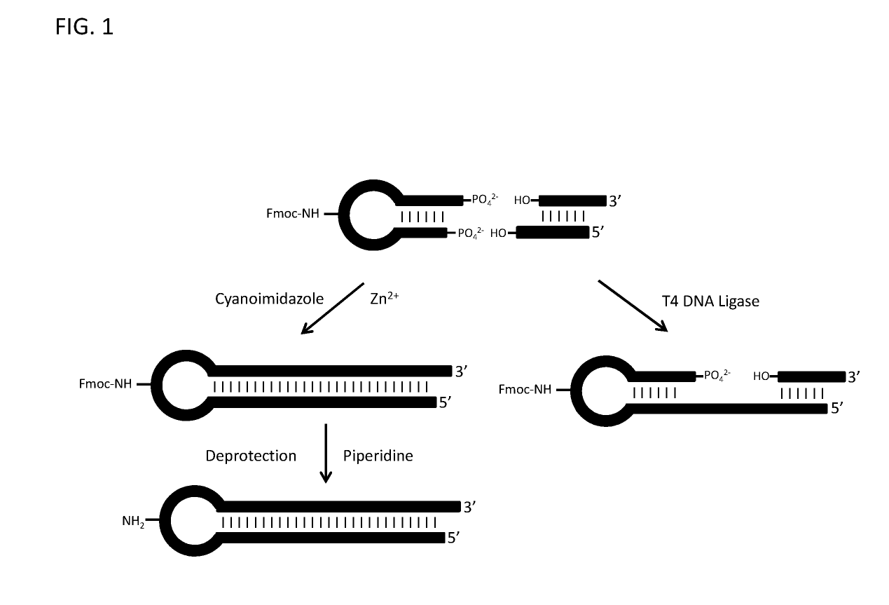

Chemical ligation can also be used to produce oligonucleotides capable of being transcribed or reverse transcribed or otherwise used as a template for a template-dependent polymerase. The efficacy of a chemical ligation technique to provide oligonucleotides capable of being transcribed or reverse transcribed may need to be tested. This efficacy can be tested by any useful method, such as liquid chromatography-mass spectrometry, RT-PCR analysis, PCR analysis, electrophoresis, and/or sequencing. In particular embodiments, chemical ligation includes the use of one or more chemical-reactive pairs to provide a spacing moiety that can be transcribed or reverse transcribed. An example of the methods of the present invention is outlined in FIG. 1 in which a double-stranded hairpin structure is utilized as a bifunctional headpiece oligonucleotide that offers sites for both chemical ligation of encoding oligonucleotide tags and a protected primary amine for the synthesis of a covalently attached encoded small-molecule. The headpiece bears both 3′- and 5′-phosphate groups, each of which may be ligated to a corresponding complementary unphosphorylated oligonucleotide using cyanoimidazole and a divalent metal ion such as Zn²⁺. The same construct may only be hemi-ligated using enzymatic ligation with T4 DNA ligase since this enzyme only supports the ligation of 5′-phosphate to 3′-hydroxyl oligonucleotides, not of 3′-phosphate to 5′-hydroxyl oligonucleotides, as indicated in FIG. 1. It was observed that unprotected primary amines reacted with the cyanoimidazole to give a guanidine adduct, however, Fmoc protection of the amine can prevent this from occurring, and the protected amine does not deprotect under the chemical ligation reaction conditions. Fmoc is readily removed with piperidine.

Reaction Conditions to Promote Enzymatic Ligation or Chemical Ligation

The methods described herein can include one or more reaction conditions that promote enzymatic or chemical ligation between the headpiece and a tag or between two tags. These reaction conditions include using modified nucleotides within the tag, as described herein; using donor tags and acceptor tags having different lengths and varying the concentration of the tags; using different types of ligases, as well as combinations thereof (e.g., CircLigase™ DNA ligase and/or T4 RNA ligase), and varying their concentration; using poly ethylene glycols (PEGs) having different molecular weights and varying their concentration; use of non-PEG crowding agents (e.g., betaine or bovine serum albumin); varying the temperature and duration for ligation; varying the concentration of various agents, including ATP, Co(NH₃)₆Cl₃, and yeast inorganic pyrophosphate; using enzymatically or chemically phosphorylated oligonucleotide tags; using 3′-protected tags; and using preadenylated tags. These reaction conditions also include chemical ligations.

The headpiece and/or tags can include one or more modified or substituted nucleotides. In preferred embodiments, the headpiece and/or tags include one or more modified or substituted nucleotides that promote enzymatic ligation, such as 2′-O-methyl nucleotides (e.g., 2′-O-methyl guanine or 2′-O-methyl uracil), 2′-fluoro nucleotides, or any other modified nucleotides that are utilized as a substrate for ligation. Alternatively, the headpiece and/or tags are modified to include one or more chemically reactive groups to support chemical ligation (e.g. an optionally substituted alkynyl group and an optionally substituted azido group). Optionally, the tag oligonucleotides are functionalized at both termini with chemically reactive groups, and, optionally, one of these termini is protected, such that the groups may be addressed independently and side-reactions may be reduced (e.g., reduced polymerization side-reactions).

As described herein, chemical ligation which results in phosphodiester, phophonate, or phosphorothioate linkages may be performed by reaction of a 5′- or 3′-phosphate, phosphonate, or phosphorothioate with a 5′- or 3′-hydroxyl group in the presence of cyanoimidazole and a divalent metal ion such as Zn²⁺.

Enzymatic ligation can include one or more ligases. Exemplary ligases include CircLigase™ ssDNA ligase (EPICENTRE Biotechnologies, Madison, Wis.), CircLigase™ II ssDNA ligase (also from EPICENTRE Biotechnologies), ThermoPhage™ ssDNA ligase (Prokazyme Ltd., Reykjavik, Iceland), T4 RNA ligase, and T4 DNA ligase. In preferred embodiments, ligation includes the use of an RNA ligase or a combination of an RNA ligase and a DNA ligase. Ligation can further include one or more soluble multivalent cations, such as Co(NH₃)₆Cl₃, in combination with one or more ligases.

Before or after the ligation step, a complex or encoded chemical entity can be purified. In some embodiments, the complex or encoded chemical entity can be purified to remove unreacted headpiece or tags that may result in cross-reactions and introduce “noise” into the encoding process. In some embodiments, the complex or encoded chemical entity can be purified to remove any reagents or unreacted starting material that can inhibit or lower the ligation activity of a ligase. For example, orthophosphate may result in lowered ligation activity. In certain embodiments, entities that are introduced into a chemical or ligation step may need to be removed to enable the subsequent chemical or ligation step. Methods of purifying the complex or encoded chemical entity are described herein. Purification of the complex may be carried out by reversible immobilization of the complex followed by purification and release prior to a subsequent step.

Enzymatic and chemical ligation can include poly ethylene glycol having an average molecular weight of more than 300 Daltons (e.g., more than 600 Daltons, 3,000 Daltons, 4,000 Daltons, 5,000, 6,000, 7,000, 8,000, 9,000, 10,000, 15,000, 20,000, 25,000, 30,000, 35,000, 40,000, or 45,000 Daltons). In particular embodiments, the poly ethylene glycol has an average molecular weight from about 3,000 Daltons to 9,000 Daltons (e.g., from 3,000 Daltons to 8,000 Daltons, from 3,000 Daltons to 7,000 Daltons, from 3,000 Daltons to 6,000 Daltons, and from 3,000 Daltons to 5,000 Daltons). In preferred embodiments, the poly ethylene glycol has an average molecular weight from about 3,000 Daltons to about 6,000 Daltons (e.g., from 3,300 Daltons to 4,500 Daltons, from 3,300 Daltons to 5,000 Daltons, from 3,300 Daltons to 5,500 Daltons, from 3,300 Daltons to 6,000 Daltons, from 3,500 Daltons to 4,500 Daltons, from 3,500 Daltons to 5,000 Daltons, from 3,500 Daltons to 5,500 Daltons, and from 3,500 Daltons to 6,000 Daltons, such as 4,600 Daltons). Poly ethylene glycol can be present in any useful amount, such as from about 25% (w/v) to about 35% (w/v), such as 30% (w/v).

### Methods for Determining the Nucleotide Sequence of a Complex

This invention features a method for determining the nucleotide sequence of a complex, such that encoding relationships may be established between the sequence of the assembled tag sequence and the structural units (or building blocks) of the chemical entity. In particular, the identity and/or history of a chemical entity can be inferred from the sequence of bases in the oligonucleotide. Using this method, a library including diverse chemical entities or members (e.g., small molecules or peptides) can be addressed with a particular tag sequence.

Any of the linkages described herein can be reversible or irreversible. Reversible linkages include photo-reactive linkages (e.g., a cyanovinylcarbozole group and thymidine) and redox linkages. Additional linkages are described herein.

In an alternative embodiment, an “unreadable” linkage can be enzymatically repaired in order to generate a readable or at least translocatable linkage. Enzymatic repair processes are well known to those skilled in the art and include, but are not limited to, pyrimidine (e.g., thymidine) dimer repair mechanisms (e.g., using a photolyase or a glycosylase (e.g., T4 pyrimidine dimer glycosylase (PDG))), base excision repair mechanisms (e.g., using a glycosylase, an apurinic/apyrimidinic (AP) endonuclease, a Flap endonuclease, or a poly ADP ribose polymerase (e.g., human apurinic/apyrimidinic (AP) endonuclease, APE 1; endonuclease III (Nth) protein; endonuclease IV; endonuclease V; formamidopyrimidine [fapy]-DNA glycosylase (Fpg); human 8-oxoguanine glycosylase 1 (α isoform) (hOGG1); human endonuclease VIII-like 1 (hNEIL1); uracil-DNA glycosylase (UDG); human single-strand selective monofunctional uracil DNA glycosylase (SMUG1); and human alkyladenine DNA glycosylase (hAAG)), which can be optionally combined with one or more endonucleases, DNA or RNA polymerases, and/or a ligases for the repair), methylation repair mechanisms (e.g., using a methyl guanine methyltransferase), AP repair mechanisms (e.g., using an apurinic/apyrimidinic (AP) endonuclease (e.g., APE 1; endonuclease III; endonuclease IV; endonuclease V; Fpg; hOGG1; and hNEIL1), which can be optionally combined with one or more endonucleases, DNA or RNA polymerases, and/or a ligases for the repair), nucleotide excision repair mechanisms (e.g., using excision repair cross-complementing proteins or excision nucleases, which can be optionally combined with one or more endonucleases, DNA or RNA polymerases, and/or a ligases for the repair), and mismatch repair mechanisms (e.g., using an endonuclease (e.g., T7 endonuclease I; MutS, MutH, and/or MutL), which can be optionally combined with one or more exonucleases, endonucleases, helicases, DNA or RNA polymerases, and/or ligases for the repair). Commercial enzyme mixtures are available to readily provide these kinds of repair mechanisms, e.g., PreCR® Repair Mix (New England Biolabs Inc., Ipswich MA), which includes Taq DNA Ligase, Endonuclease IV, Bst DNA Polymerase, Fpg, Uracil-DNA Glycosylase (UDG), T4 PDG (T4 Endonuclease V), and Endonuclease VIII.

### Methods for Tagging Encoded Libraries

This invention features a method for operatively associating oligonucleotide tags with chemical entities, such that encoding relationships may be established between the sequence of the tag and the structural units (or building blocks) of the chemical entity. In particular, the identity and/or history of a chemical entity can be inferred from the sequence of bases in the oligonucleotide. Using this method, a library including diverse chemical entities or members (e.g., small molecules or peptides) can be encoded with a particular tag sequence.

Generally, these methods include the use of a headpiece, which has at least one functional group that may be elaborated chemically and at least one functional group to which a oligonucleotide tag may be bound (or ligated). Binding can be effectuated by any useful means, such as by enzymatic binding (e.g., ligation with one or more of an RNA ligase and/or a DNA ligase) or by chemical binding (e.g., by a substitution reaction between two functional groups, such as a nucleophile and a leaving group).

To create numerous chemical entities within the library, a solution containing the headpiece can be divided into multiple aliquots and then placed into a multiplicity of physically separate compartments, such as the wells of a multiwell plate. Generally, this is the “split” step. Within each compartment or well, successive chemical reaction and ligation steps are performed with a oligonucleotide tag within each aliquot. The relationship between the chemical reaction conditions and the sequence of the—tag are associated. The reaction and ligation steps may be performed in any order. Then, the reacted and ligated aliquots are combined or “pooled,” and optionally purification may be performed at this point. Purification may be performed by reversible immobilization of the complex, removal of the solvent and any reagents/containments, followed by release of the complex prior to a subsequent step. These split and pool steps can be optionally repeated.

Next, the library can be tested and/or selected for a particular characteristic or function, as described herein. For example, the mixture of tagged chemical entities can be separated into at least two populations, where the first population is enriched for members that bind to a particular biological target and the second population that is less enriched (e.g., by negative selection or positive selection). The first population can then be selectively captured (e.g., by eluting on a column providing the target of interest or by incubating the aliquot with the target of interest) and, optionally, further analyzed or tested, such as with optional washing, purification, negative selection, positive selection, or separation steps. Finally, the chemical histories of one or more members (or chemical entities) within the selected population can be determined by the sequence of the operatively linked oligonucleotide. Upon correlating the sequence with encoded library members chemical history, this method can identify the individual members of the library with the selected characteristic (e.g., an increased tendency to bind to the target protein and thereby elicit a therapeutic effect). For further testing and optimization, candidate therapeutic compounds may then be prepared by synthesizing the identified library members with or without their associated oligonucleotide tags.

The methods described herein can include any number of optional steps to diversify the library or to interrogate the members of the library. For any tagging method described herein, successive “n” number of tags can be added with additional “n” number of ligation, separation, and/or phosphorylation steps. Exemplary optional steps include restriction of library member-associated encoding oligonucleotides using one or more restriction endonucleases; repair of the associated encoding oligonucleotides, e.g., with any repair enzyme, such as those described herein; ligation of one or more adapter sequences to one or both of the termini for library member-associated encoding oligonucleotides, e.g., such as one or more adapter sequences to provide a priming sequence for amplification and sequencing or to provide a label, such as biotin, for immobilization of the sequence; reverse-transcription or transcription, optionally followed by reverse-transcription, of the assembled tags in the complex using a reverse transcriptase, transcriptase, or another template-dependent polymerase; amplification of the assembled tags in the complex using, e.g., PCR; generation of clonal isolates of one or more populations of assembled tags in the complex, e.g., by use of bacterial transformation, emulsion formation, dilution, surface capture techniques, etc.; amplification of clonal isolates of one or more populations of assembled tag in the complex, e.g., by using clonal isolates as templates for template-dependent polymerization of nucleotides; and sequence determination of clonal isolates of one or more populations of assembled tags in the complex, e.g., by using clonal isolates as templates for template-dependent polymerization with fluorescently labeled nucleotides with reversible terminator chemistry. Additional methods for amplifying and sequencing the oligonucleotide tags are described herein.

These methods can be used to identify and discover any number of chemical entities with a particular characteristic or function, e.g., in a selection step. The desired characteristic or function may be used as the basis for partitioning the library into at least two parts with the concomitant enrichment of at least one of the members or related members in the library with the desired function. In particular embodiments, the method comprises identifying a small drug-like library member that binds or inactivates a protein of therapeutic interest. In another embodiment, a sequence of chemical reactions is designed, and a set of building blocks is chosen so that the reaction of the chosen building blocks under the defined chemical conditions will generate a combinatorial plurality of molecules (or a library of molecules), where one or more molecules may have utility as a therapeutic agent for a particular protein. For example, the chemical reactions and building blocks are chosen to create a library having structural groups commonly present in kinase inhibitors. In any of these instances, the oligonucleotide tags encode the chemical history of the library member and in each case a collection of chemical possibilities may be represented by any particular tag combination.

In one embodiment, the library of chemical entities, or a portion thereof, is contacted with a biological target under conditions suitable for at least one member of the library to bind to the target, followed by removal of library members that do not bind to the target, and analyzing the one or more oligonucleotide tags associated with the target. This method can optionally include amplifying the tags by methods known in the art. Exemplary biological targets include enzymes (e.g., kinases, phosphatases, methylases, demethylases, proteases, and DNA repair enzymes), proteins involved in protein:protein interactions (e.g., ligands for receptors), receptor targets (e.g., GPCRs and RTKs), ion channels, bacteria, viruses, parasites, DNA, RNA, prions, and carbohydrates.

In another embodiment, the chemical entities that bind to a target are not subjected to amplification but are analyzed directly. Exemplary methods of analysis include microarray analysis, including evanescent resonance photonic crystal analysis; bead-based methods for deconvoluting tags (e.g., by using his-tags); label-free photonic crystal biosensor analysis (e.g., a BIND® Reader from SRU Biosystems, Inc., Woburn, Mass.); or hybridization-based approaches (e.g. by using arrays of immobilized oligonucleotides complementary to sequences present in the library of tags).

In addition, chemical-reactive pairs (or functional groups) can be readily included in solid-phase oligonucleotide synthesis schemes and will support the efficient chemical ligation of oligonucleotides. In addition, the resultant ligated oligonucleotides can act as templates for template-dependent polymerization with one or more polymerases. Accordingly, any of the binding steps described herein for tagging encoded libraries can be modified to include one or more of enzymatic ligation and/or chemical ligation techniques. Exemplary ligation techniques include enzyme ligation, such as use of one of more RNA ligases and/or DNA ligases; and chemical ligation, such as use of chemical-reactive pairs (e.g., a pair including optionally substituted alkynyl and azido functional groups).

Furthermore, one or more libraries can be combined in a split-and-mix step. In order to permit mixing of two or more libraries, the library member may contain one or more library-identifying sequences, such as in a library-identifying tag, in a ligated tag, or as part of the headpiece sequence, as described herein.

Methods for Encoding Chemical Entities within a Library

The methods of the invention can be used to synthesize a library having a diverse number of chemical entities that are encoded by oligonucleotide tags. Examples of building blocks and encoding DNA tags are found in U.S. Patent Application Publication No. 2007/0224607, the building blocks and tags of which are hereby incorporated by reference.

Each chemical entity is formed from one or more building blocks and optionally a scaffold. The scaffold serves to provide one or more diversity nodes in a particular geometry (e.g., a triazine to provide three nodes spatially arranged around a heteroaryl ring or a linear geometry).

The building blocks and their encoding tags can be added directly or indirectly (e.g., via a spacer) to the headpiece to form a complex. When the headpiece includes a spacer, the building block or scaffold is added to the end of the spacer. When the spacer is absent, the building block can be added directly to the headpiece or the building block itself can include a spacer that reacts with a functional group of the headpiece. Exemplary spacers and headpieces are described herein.

The scaffold can be added in any useful way. For example, the scaffold can be added to the end of the spacer or the headpiece, and successive building blocks can be added to the available diversity nodes of the scaffold. In another example, building block Aₙ is first added to the spacer or the headpiece, and then the diversity node of scaffold S is reacted with a functional group in building block Aₙ. Oligonucleotide tags encoding a particular scaffold can optionally be added to the headpiece or the complex. For example, Sₙ is added to the complex in n reaction vessels, where n is an integer more than one, and tag Sₙ (i.e., tag S₁, S₂, Sₙ₋₁, Sₙ) is bound to the functional group of the complex.

Building blocks can be added in multiple, synthetic steps. For example, an aliquot of the headpiece, optionally having an attached spacer, is separated into n reaction vessels, where n is an integer of two or greater. In the first step, building block Aₙ is added to each n reaction vessel (i.e., building block A₁, A₂, . . . Aₙ₋₁, Aₙ is added to reaction vessel 1, 2, . . . n-1, n), where n is an integer and each building block Aₙ is unique. In the second step, scaffold S is added to each reaction vessel to form an Aₙ-S complex. Optionally, scaffold Sₙ can be added to each reaction vessel to from an Aₙ-Sₙ complex, where n is an integer of more than two, and each scaffold Sₙ can be unique. In the third step, building block Bₙ is to each n reaction vessel containing the Aₙ-S complex (i.e., building block B₁, B₂, . . . Bₙ₋₁, Bₙ is added to reaction vessel 1, 2, . . . n-1, n containing the A₁-S, A₂-S, . . . Aₙ₋₁-S, Aₙ-S complex), where each building block Bₙ is unique. In further steps, building block Oₙ can be added to each n reaction vessel containing the Bₙ-Aₙ-S complex (i.e., building block C₁, C₂, . . . Cₙ₋₁, Cₙ is added to reaction vessel 1, 2, . . . n-1, n containing the B₁-A₁-S . . . Bₙ-Aₙ-S complex), where each building block Cₙ is unique. The resulting library will have n³ number of complexes having n³ tags. In this manner, additional synthetic steps can be used to bind additional building blocks to further diversify the library.

After forming the library, the resultant complexes can optionally be purified and subjected to a polymerization or ligation reaction, e.g., to a tailpiece. This general strategy can be expanded to include additional diversity nodes and building blocks (e.g., D, E, F, etc.). For example, the first diversity node is reacted with building blocks and/or S and encoded by an oligonucleotide tag. Then, additional building blocks are reacted with the resultant complex, and the subsequent diversity node is derivatized by additional building blocks, which is encoded by the primer used for the polymerization or ligation reaction.

To form an encoded library, oligonucleotide tags are added to the complex after or before each synthetic step. For example, before or after the addition of building block Aₙ to each reaction vessel, tag Aₙ is bound to the functional group of the headpiece (i.e., tag A₁, A₂, . . . Aₙ₋₁, Aₙ is added to reaction vessel 1, 2, . . . n-1, n containing the headpiece). Each tag Aₙ has a distinct sequence that correlates with each unique building block Aₙ, and determining the sequence of tag Aₙ provides the chemical structure of building block Aₙ. In this manner, additional tags are used to encode for additional building blocks or additional scaffolds.

Furthermore, the last tag added to the complex can either include a primer-binding sequence or provide a functional group to allow for binding (e.g., by ligation) of a primer-binding sequence. The primer-binding sequence can be used for amplifying and/or sequencing the oligonucleotides tags of the complex. Exemplary methods for amplifying and for sequencing include polymerase chain reaction (PCR), linear chain amplification (LCR), rolling circle amplification (RCA), or any other method known in the art to amplify or determine nucleic acid sequences.

Using these methods, large libraries can be formed having a large number of encoded chemical entities. For example, a headpiece is reacted with a spacer and building block Aₙ, which includes 1,000 different variants (i.e., n=1,000). For each building block Aₙ, a DNA tag Aₙ is ligated or primer extended to the headpiece. These reactions may be performed in a 1,000-well plate or 10×100 well plates. All reactions may be pooled, optionally purified, and split into a second set of plates. Next, the same procedure may be performed with building block Bₙ, which also include 1,000 different variants. A DNA tag Bₙ may be ligated to the Aₙ-headpiece complex, and all reactions may be pooled. The resultant library includes 1,000×1,000 combinations of Aₙ×Bₙ (i.e., 1,000,000 compounds) tagged by 1,000,000 different combinations of tags. The same approach may be extended to add building blocks Cₙ, Dₙ, Eₙ, etc. The generated library may then be used to identify compounds that bind to the target. The structure of the chemical entities that bind to the library can optionally be assessed by PCR and sequencing of the DNA tags to identify the compounds that were enriched.

This method can be modified to avoid tagging after the addition of each building block or to avoid pooling (or mixing). For example, the method can be modified by adding building block Aₙ to n reaction vessels, where n is an integer of more than one, and adding the identical building block B₁ to each reaction well. Here, B₁ is identical for each chemical entity, and, therefore, an oligonucleotide tag encoding this building block is not needed. After adding a building block, the complexes may be pooled or not pooled. For example, the library is not pooled following the final step of building block addition, and the pools are screened individually to identify compound(s) that bind to a target. To avoid pooling all of the reactions after synthesis, a binding assay e.g. ELISA, SPR, ITC, Tm shift, SEC or similar, for example, may be used to monitor binding on a sensor surface in high throughput format (e.g., 384 well plates and 1,536 well plates). For example, building block Aₙ may be encoded with DNA tag Aₙ, and building block Bₙ may be encoded by its position within the well plate. Candidate compounds can then be identified by using a binding assay (e.g., ELISA, SPR, ITC, Tm shift, SEC or similar) and by analyzing the Aₙ tags by sequencing, microarray analysis and/or restriction digest analysis. This analysis allows for the identification of combinations of building blocks Aₙ and Bₙ that produce the desired molecules.

The method of amplifying can optionally include forming a water-in-oil emulsion to create a plurality of aqueous microreactors. The reaction conditions (e.g., concentration of complex and size of microreactors) can be adjusted to provide, on average, a microreactor having at least one member of a library of compounds. Each microreactor can also contain the target, a single bead capable of binding to a complex or a portion of the complex (e.g., one or more tags) and/or binding the target, and an amplification reaction solution having one or more necessary reagents to perform nucleic acid amplification. After amplifying the tag in the microreactors, the amplified copies of the tag will bind to the beads in the microreactors, and the coated beads can be identified by any useful method.

Once the building blocks from the first library that bind to the target of interest have been identified, a second library may be prepared in an iterative fashion. For example, one or two additional nodes of diversity can be added, and the second library is created and sampled, as described herein. This process can be repeated as many times as necessary to create molecules with desired molecular and pharmaceutical properties.

Various ligation techniques can be used to add the scaffold, building blocks, spacers, linkages, and tags. Accordingly, any of the binding steps described herein can include any useful ligation technique or techniques. Exemplary ligation techniques include enzymatic ligation, such as use of one of more RNA ligases and/or DNA ligases, as described herein; and chemical ligation, such as use of chemical-reactive pairs, as described herein.

### EXAMPLES

### Example 1

### Preparation of the Components for the Chemical Ligation (Double-Stranded Headpiece and Double-Stranded Tag)

Headpiece HP006, chemically phosphorylated at its 5′ end SEQ ID NO: 1 -(p)CCTGTGTTZTTCACGGCCT, where Z stands for C6-amino dT modification, was acquired from Biosearch Inc. HP006 was then modified by DMT-MM acylation using Fmoc-NH-PEG4-CH2CH2COOH (Chem Pep Inc) using the following procedure.

50 equivalents of Fmoc-NH-PEG4-CH2CH2COOH (Chem Pep Inc) were dissolved in DMA (Dimethyl acetamide, Acros) and added to 1 equivalent of HP006 dissolved in 0.5 M Borate buffer pH 9.5 together with 50 equivalents of DMT-MM (4-(4,6-Dimethoxy-1,3,5-triazin-2-yl)-4-methylmorpholinium Chloride, Acros), freshly dissolved in water. The reaction was allowed to proceed for 2-4 hrs, followed by a second addition of 50 equivalents of Fmoc-NH-PEG4-CH2CH2COOH and of 50 equivalents of DMT-MM and the reaction was then allowed to proceed overnight. Completion of the reaction was monitored by LCMS.

The product was ethanol precipitated and desalted by size exclusion spin filtration using 3,000 MW cut-off centrifugal spin filters (Millipore). LCMS of the product confirmed the MW as 6,803.3 (Calcd 6,802.5).

Oligonucleotides TagZA1+_deltaC_5OH: SEQ ID NO: 2-5′ CATCAAGACCCAGAAAG-3′, TagZB_CNIm_bot3OH; SEQ ID NO: 3-5′-(p)TCTGGGTCTTGATGGCTATCC-3′ (chemically phosphorylated at 5′ terminus), PrA_CNIm_bot5P; SEQ ID NO: 4-5′-(p)TGGCTGAGG-3′ (chemically phosphorylated at 5′ terminus) and PrA_top_extraC_3P; SEQ ID NO: 5-5′-(p)CAGCCAGGATAGC(p)-3′ (chemically phosphorylated at both 5′ and 3′ termini) were acquired from IDT DNA.

Oligos tagZA1+_deltaC and TagZB_CNIm_bot3OH were then dissolved to a 2 mM final concentration in water and combined at equimolar ratio to make 1 mM solution of double stranded TagZA.

Oligos PrA_CNIm_bot5P and PrA_top_extraC_3P were also dissolved to a 2 mM final concentration in water and combined at equimolar ratio to make 1 mM solution of double stranded “CNIm-PrA”.

Fmoc-amino-PEG4-HP006 was then enzymatically ligated to one equivalent of double stranded CNIm-PrA using T4 DNA ligase and a standard ligation protocol. The resulting oligo, (Fmoc-amino-PEG4-HP013), was ethanol precipitated and desalted using Illustra NAP-5 columns (GE Healthcare Life Science). LCMS confirmed MW 13,772 (calcd 13,770.7).

### Example 2

### The Chemical Ligation of a Double-Stranded Headpiece to a Double-Stranded Tag

Fmoc-amino-PEG4-HP013 and double-stranded TagZA oligonucleotides were dissolved to a final concentration of 0.33 mM in 80 mM MES buffer pH 6.0, containing 800 mM NaCl, and 8 mM ZnCl₂. 1-Cyanoimidazole was freshly dissolved to 1 M in DMF and 1-2 additions were made to the reaction over a 12-hour period to a final concentration of 1-cyanoimidazole of 150 mM. The reaction was then incubated at 4° C. overnight.

The completed reaction was analyzed by denaturing gel electrophoresis as well as by LCMS. The samples were then resolved on a 15% denaturing analytical TBE-8M Urea gel and visualized by UV shadowing over a TLC plate with a fluorescent dye (254 nm). LCMS confirmed formation of the double-stranded ligated product with MW 25,417.3 (calc 25,415.3) with ˜70% conversion. Additional products with MW 20,254.7 and 18,935.4 were observed, which corresponded to either the (hemi-ligated) top or bottom strand ligation products.

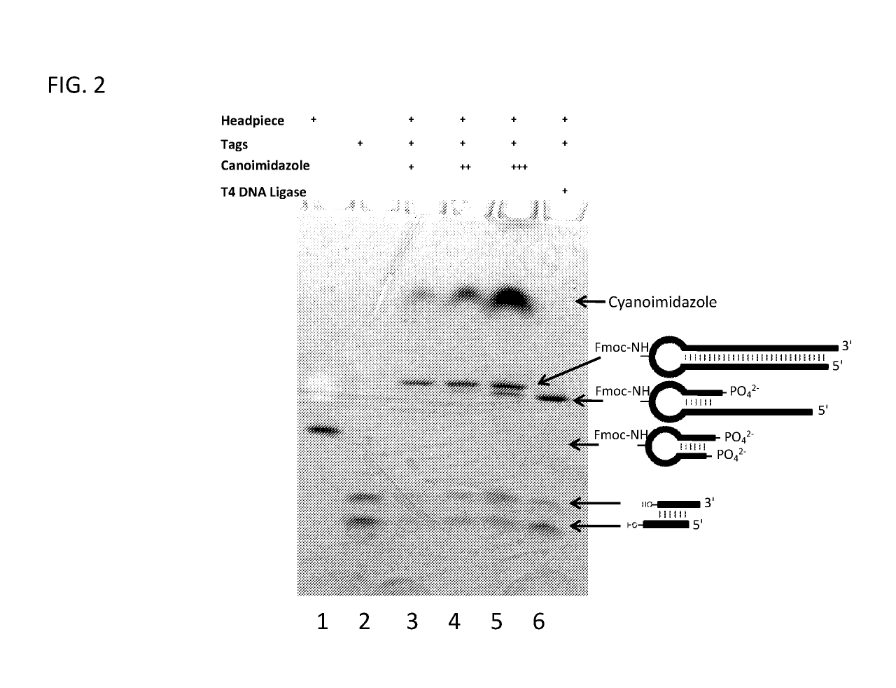

Analytical gel electrophoresis of the chemical ligation products with a 15% TBE-8M Urea denaturing gel is shown in FIG. 2:

1—Starting material—Fmoc-amino-PEG4-HP013

2—dsTag ZA, which is an equimolar mixture of tagZA1_deltaC_5OH and TagZA1+_CNIm_bot3OH

3, 4, 5—Cyanoimidazole ligation reactions

6—Enzymatic ligation control (T4 DNA ligase) ligates only the bottom strand, the junction between 3′ OH and 5′phosphate; the junction between 3′phosphate and 5′ OH is not be ligated by this enzyme.

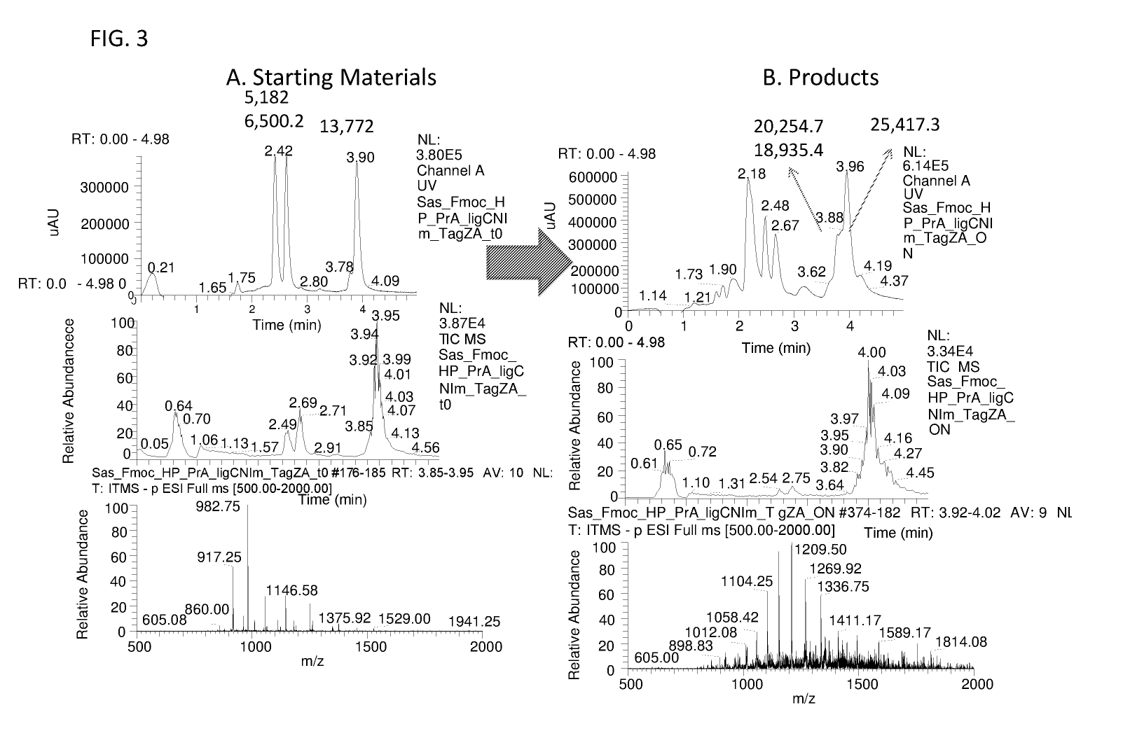

LCMS of the chemical ligation products are shown in FIG. 3. (In each panel—top UV (260 nm) LC trace, middle—TIC, bottom—mass spectrum)
A.—Starting materials: mixture of double stranded TagZA (MW 5,182 and 6,500.2 Da) as well as Fmoc-amino-PEG4-HP013 (13,772).
B—Products of the chemical ligation reaction: doubly ligated: MW 25,417.3 (calc 25,415.3). Hemi-ligated (either top or bottom strand) products: MW 20,254.7 and 18,935.4.

### Example 3

### Fmoc Deprotection of the Chemical Ligation Reaction Products

Products of the 1-cyanoimidazole ligation reaction were ethanol precipitated, dissolved in water and deprotected by incubation in 10% piperidine for 2 hours at room temperature. Following this deprotection step, the material was purified on a 15% TBE-8M urea gel. The LC-MS performed on the purified sample confirmed the presence of the deprotected amino-PEG4-HP013-TagZA (MW 25,192.4, calc 25,193.2) as well as the two hemi-ligated deprotected products (MW 18,738.6 and 20,029.3).

Integration of the LC trace gives the relative yields of the full length product at 64%, while the hemi-ligated products at roughly 18% each. The efficiency of the ligation per strand can be estimated at 83%.

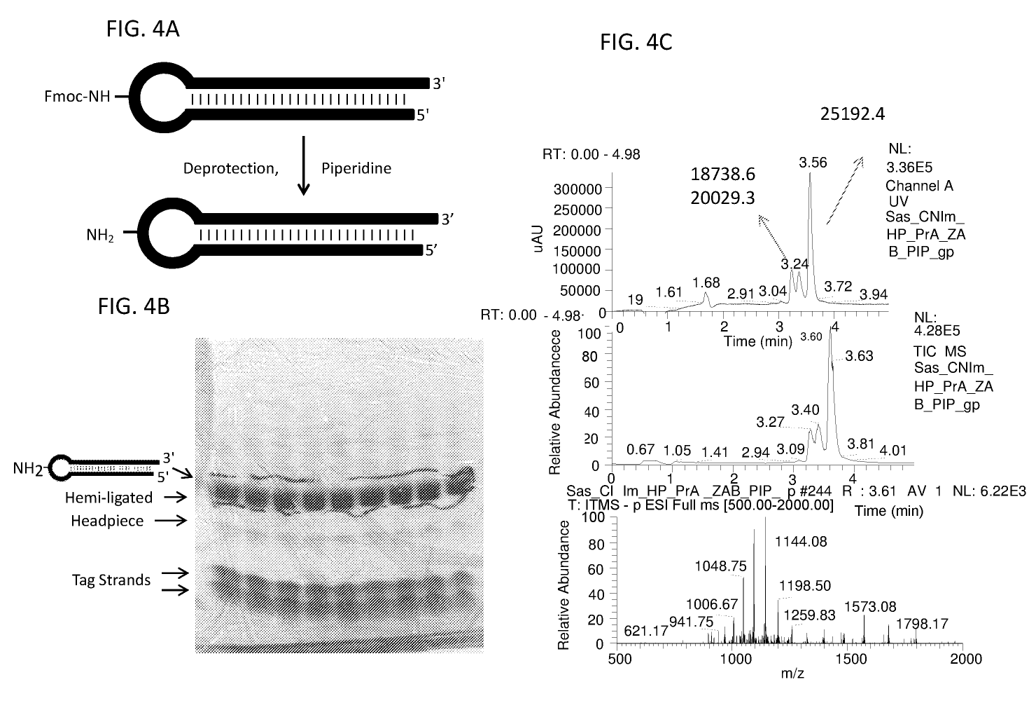

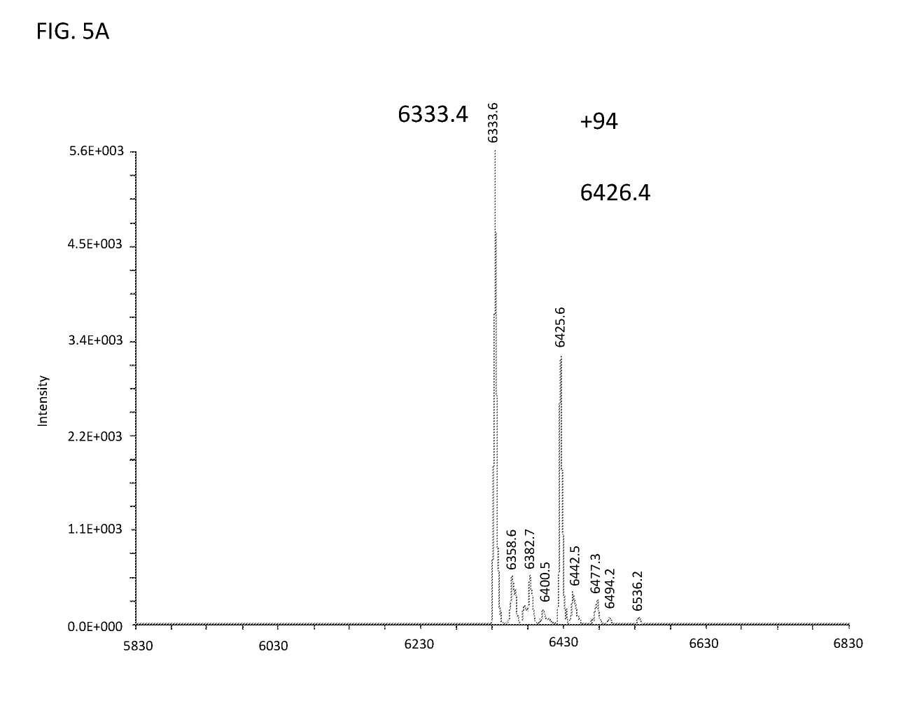

Schematics of the amino deprotection by piperidine are shown in FIG. 4A. Gel purification of the ligation reaction products: 15% TBE-Urea gel, UV shadowing is shown in FIG. 4B. LCMS analysis of the purified material is shown in FIG. 4C. Full-length ligation product at MW 25,192.4 Da, hemi-ligated products at MW 18,738.6 and 20,029.3 Da.

### Example 4

### Illustration of the Necessity for Amino-Group Protection with Fmoc

HP006, which features an amino-C6 linker at T in the loop, as described above, was incubated in the reaction mixture with 1-cyanoimidazole for 12 hours at 4 ° C. Following the incubation, HP006 was ethanol-precipitated, incubated for 2 hrs at room temperature in 10% piperidine and ethanol precipitated again.

LCMS analysis of this material demonstrated that there are two products in the mixture, the MW 6,333.4 Da HP006 and a MW 6,426.4 reaction product (30-40% conversion). The addition of 94 Da corresponds to the formation of an N-imidazole guanidine derivative of HP006. Fmoc protection of the amino group completely eliminates this unwanted reaction.

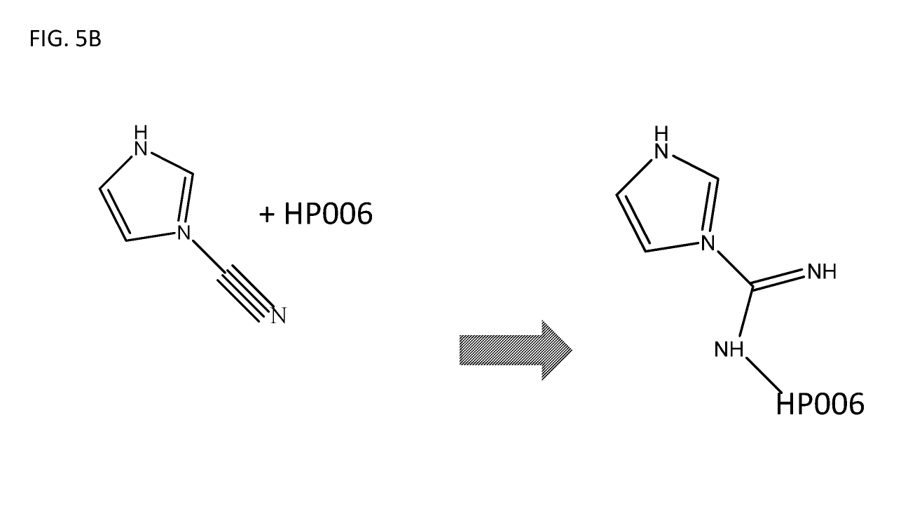

A deconvoluted mass spectrum of the product of the reaction of HP006 with 1-cyanoimidazole is shown in FIG. 5A. MW 6,333.4 Da corresponds to unmodified HP006, MW 6,426.4 corresponds to an N-imidazole guanidine derivative of HP006.

A schematic of the generation of the N-imidazole guanidine derivative of HP006 is shown in FIG. 5B.

### Example 5

### Chemical Ligation with Alternative Divalent Metal Ions

Cyanoimidazole-mediated chemical ligation was performed as described above with the substitution of 8 mM of alternative divalent metals. Significant ligation yields were observed with CoCl₂ (30% full-length product, 70% of hemi-ligated products), MnCl₂ (75% full-length product, 25% of hemi-ligated products and ZnCl₂ (60% of full-length product with 30% of hemi-ligated products). Soluble divalent salts of lead, magnesium, tin and copper produced no significant ligation.

### Example 6

### Chemical Ligation with Alternative Flanking Nucleotides

The following chemically phosphorylated oligonucleotides were acquired from IDT DNA Top strand, pair 1:

PrA_top: SEQ ID NO: 6-
5′-(p)CAGCCAGGATAG-3′;
Tag_ZA1+:
5′-(p)CCATCAAGACCCAGAAAG-3′;
Top strand, pair 2:
PrA_top_extraC_3P:
5′-(p)CAGCCAGGATAGCp-3′;
tagZA1_deltaC_5OH:
5′-CATCAAGACCCAGAAAG-3′
(overlap sequences in bold)
Bottom strand, pair A:
PrA_CNlm_bot5P:
5′-pTGGCTGAGG-3′;
TagZB_CNlm_bot3OH:
5′-pTCTGGGTCTTGATGGCTATCC-3′
Bottom strand, pair B:
PrA_CNlm_bot5OH:
5′-TGGCTGAGG-3′:
TagZB_CNlm_bot3P:
5′-pTCTGGGTCTTGATGGCTATCCp-3′

Four combinations of oligonucleotides were tested for the efficiency of 1-cyanoimidazole ligation, as shown in Table 2. While bottom strands demonstrated consistently high yields of ligation with both 6- and 7-nucleotide overlaps (greater than 80%), and in both tested combinations of flanking nucleotides (C to C and C to T), the top strand ligations were apparently dependent on the identity of the flanking nucleotides, e.g. ligation of C to G was inefficient, whereas C to C junctions ligated at high yield.

TABLE 2
Summary of ligation junction designs
and yields of chemical ligation

| | Overlap | Ligation | Bottom strand | Relative ligation |
|---|---|---|---|---|
| | length | junction | ligation | conversion |
| Reaction | (nts) | (top strand) | junction | (top strand) |
| 1-A | 6 | C-3′ + 5′pG | C-3′ + 5′pT | 20% |
| 1-B | 6 | C-3′ + 5′pG | Cp-3′ + 5′-T | 25% |
| 2-A | 7 | Cp-3′ + 5′-C | C-3′ + 5′pT | 90% |
| 2-B | 7 | Cp-3′ + 5′-C | Cp-3′ + 5′-T | 95% |

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

1. A method of producing an encoded chemical entity, said method comprising:
(a) providing a headpiece comprising a first functional group and a second functional group;
(b) binding said first functional group of said headpiece to a component of said chemical entity, wherein said headpiece is directly connected to said component or said headpiece is indirectly connected to said component by a bifunctional spacer;
(c) ligating said second functional group of said headpiece to a first oligonucleotide tag via chemical ligation to form an encoded chemical entity, wherein said chemical ligation generates a phosphodiester, phosphonate, or phosphorothioate linkage;
wherein steps (b) and (c) can be performed in any order and wherein said first oligonucleotide tag encodes for the binding reaction of said step (b),
thereby producing an encoded chemical entity.

2. The method of claim 1, wherein said chemical ligation generates a phosphodiester linkage.

3. The method of claim 1 or 2, wherein said headpiece comprises a double-stranded oligonucleotide, a single-stranded oligonucleotide, or a hairpin oligonucleotide.

4. The method of claim 3, wherein said headpiece comprises a double stranded oligonucleotide or a hairpin oligonucleotide.

5. The method of claim 4, wherein said headpiece comprises a third functional group.

6. The method of claim 5, wherein said method further comprises (d) ligating said third functional group of said headpiece to a second oligonucleotide tag via chemical ligation, wherein said chemical ligation generates a phosphodiester, phosphonate, or phosphorothioate linkage.

7. The method of claim 5, wherein said method further comprises (d) ligating said third functional group of said headpiece to a second oligonucleotide tag, wherein said ligation is not via chemical ligation that generates a phosphodiester, phosphonate, or phosphorothioate linkage

8. The method of claim 2, wherein said headpiece comprises a phosphate at the 5′-terminus and/or the 3′-terminus.

9. The method of claim 2, wherein said chemical ligation comprises the ligation of a 5′- or 3′-phosphate on said headpiece to a 5′- or 3′-hydroxyl oligonucleotide.

10. The method of claim 9, wherein said chemical ligation comprises the ligation of a 5′-phosphate on said headpiece to a 3′-hydroxyl oligonucleotide and/or a 3′-phosphate on said headpiece to a 5′-hydroxyl oligonucleotide.

11. The method of claim 10, wherein said chemical ligation comprises the simultaneous ligation of a 5′-phosphate on said headpiece to a 3′-hydroxyl oligonucleotide and a 3′-phosphate on said headpiece to a 5′-hydroxyl oligonucleotide.

12. The method of claim 8, wherein said chemical ligation comprises the use of cyanoimidazole.

13. The method of claim 12, wherein said chemical ligation further comprises the use of a divalent metal source.

14. The method of claim 13, wherein said divalent metal source is a soluble Zn²⁺ source.

15. The method of claim 14, wherein said soluble Zn²⁺ source is ZnCl₂.

16. The method of claim 1, wherein said headpiece is indirectly connected to said component by a bifunctional spacer.

17. The method of claim 1, wherein said headpiece is directly connected to said component.

18. A library comprising one or more chemical entities produced by the method of claim 1.

19. The library of claim 18, wherein said library comprises a plurality of headpieces.

20. The library of claim 18, wherein each chemical entity is different.

21. A method of screening a plurality of chemical entities, said method comprising:
(a) contacting a target with an encoded chemical entity prepared by a method of claim 1; and
(b) selecting one or more encoded chemical entities having a predetermined characteristic for said target, as compared to a control, thereby screening a plurality of said chemical entities.

22. The method of claim 21, where said predetermined characteristic comprises increased binding for said target, as compared to a control.
